(19) United States
(12) Patent Application Publication
(10) Pub. No.: US 2023/0003731 A1
(43) Pub. Date: Jan. 5, 2023

(54) PROSTATE CANCER BIOMARKERS

(71) Applicant: GLYCOSCOREDX LIMITED, Newcastle Upon Tyne (GB)

(72) Inventors: Jennifer MUNKLEY, Newcastle upon Tyne (GB); Emma SCOTT, Newcastle upon Tyne (GB)

(21) Appl. No.: 17/780,508

(22) PCT Filed: Nov. 27, 2020
(86) PCT No.: PCT/GB2020/053050

§ 371 Date: May 26, 2022

(30) Foreign Application Priority Data
Nov. 29, 2019 (GB) 1917428.3
Apr. 30, 2020 (GB) 2006423.4
Jun. 22, 2020 (GB) 2009505.5

(51) Int. Cl. G01N 33/574; G01N 33/543

(52) U.S. Cl. CPC G01N 33/57434; G01N 33/54386; G01N 2800/50; G01N 2800/52

(57) ABSTRACT

The present invention provides ST6GAL1, GALNT7, FUT8 and GCNT1 as novel biological fluid (e.g. blood or urine) biomarkers for prostate cancer. Methods for diagnosing prostate cancer or the risk of developing prostate cancer, or for monitoring prostate cancer progression (including prostate cancer relapse) and methods for treatment of prostate cancer are also provided. The invention also provides methods for determining the therapeutic effect of appropriate treatment regimens for prostate cancer or determining a subject's compliance or adherence with a prescribed treatment regimen for prostate cancer. Corresponding kits, assay devices and uses are also provided.

The present invention provides novel biomarkers for prostate cancer. Methods for diagnosing prostate cancer or the risk of developing prostate cancer, or for monitoring prostate cancer progression (including prostate cancer relapse) and methods for treatment of prostate cancer are also provided. The invention also provides methods for determining the therapeutic effect of appropriate treatment regimens for prostate cancer or determining a subject's compliance or adherence with a prescribed treatment regimen for prostate cancer. Corresponding kits, assay devices and uses are also provided.

### BACKGROUND

Prostate cancer is the most common cancer in men in the UK. It develops slowly and can go undetected for many years. It has been estimated that the economic burden of the disease in the UK exceeds £269 million. This cost is expected to increase due to the rising prevalence of the disease, attributed to an ageing population.

Almost 90% of prostate cancers are detected by means of screening. The current method of diagnosis relies on a combination of a Prostate Specific Antigen (PSA) blood test and tissue biopsy. The PSA blood test fails to detect 15% of prostate cancers and although high levels of PSA can be a sign of prostate cancer, PSA levels may also be raised for other reasons, such as the presence of a urinary track invention (UTI), undertaking vigorous exercise, or taking certain medication. Testing PSA levels only is therefore prone to under- and over-diagnosis (aggressive cancers can be missed and false positive rates can be up to 75%).

PSA levels are also unable to differentiate between benign and aggressive forms of the disease, and therefore are not informative for disease prognosis or treatment selection. It is estimated that less than 15% of men diagnosed using PSA go on to develop fatal prostate cancer. The main issue with the PSA test is that it has a very low PPV (30-65%), which is caused by a number of factors, most important of which is benign prostatic hyperplasia (BPH). Patients with BPH are unlikely to develop prostate cancer. At the moment, the only way to know for sure if a man has prostate cancer is to undergo a tissue biopsy, an invasive procedure that is expensive, painful, can lead to infections, and only provides a snapshot of the tumour. During the procedure, a doctor or nurse uses a thin needle to take small samples of tissue from the prostate (10 to 12 small pieces of tissue from different areas of the prostate). The tissue is then looked at under a microscope to check for cancer. If prostate cancer is detected the results will also show how aggressive it is based on its Gleason score.

There is an urgent unmet clinical need to improve the diagnosis of prostate cancer and to develop tests that distinguish between slow growing tumours which don't need treating, and aggressive prostate cancers that need urgent intervention.

### BRIEF SUMMARY OF THE DISCLOSURE

Glycosylation enzymes are intracellular enzymes that are predominantly located in the Golgi or the endoplasmic reticulum of a cell (Stanley P, Golgi glycosylation; Cold Spring Harb Perspect Biol. 2011 Apr. 1; 3(4)). They are responsible for attaching glycans to proteins or other organic molecules via glycosylation, a co-translational and post-translational modification that occurs during protein synthesis within the cell.

It has previously been observed that glycans change dramatically in prostate cancer and that they have key roles in driving tumour growth and spread. Furthermore, RNA levels of eight glycosylation enzymes have previously been shown to increase in clinical prostate cancer tissue (Munkley et al. 2016). Intracellular glycosylation processes therefore appear to be affected in prostate cancer.

The invention is based on the surprising finding that in men with prostate cancer a specific subset of intracellular glycosylation enzymes are secreted from cells into biological fluids such as blood and urine. The presence of these enzymes in biological fluids is unexpected, as they are not secreted out of the cell under normal circumstances. The levels of these glycosylation enzymes in biological fluids such as blood and urine may be used as biomarkers for prostate cancer, either individually, or in combination. Testing biological fluids (as opposed to tissue biopsy) is advantageous, as it does not require invasive, painful procedures. Furthermore, analysing circulating biomarkers provide a more comprehensive view of the tumour than a needle biopsy.

The inventors have tested for the presence of several glycosylation enzymes in urine or blood obtained from men with prostate cancer. Surprisingly, they found that changes in the protein levels of GCNT1, GALNT7, FUT8 and ST6GAL1 can be detected in urine or blood samples from men with prostate cancer, whereas other glycosylation enzymes (such as CSGALNACT1) could not be detected in these samples (data not shown). Assays that detect the presence of one or more of these proteins in such biological fluids may therefore be useful for prostate cancer diagnosis, prognosis and patient stratification for treatment.

The data presented herein shows that detection of GCNT1, GALNT7 and/or ST6GAL1 protein in blood or urine provides a means for distinguishing between prostate cancer and control samples (e.g. samples with benign prostatic hyperplasia (BPH)) more accurately than PSA testing. This particular combination of three markers (GCNT1, GALNT7 and ST6GAL1; referred to as “Glycoscore” herein) is therefore particularly beneficial when testing for prostate cancer per se. The data presented herein show that detection of FUT8 protein in such biological fluids can also be used to distinguish between prostate cancer and control samples (e.g. samples with benign prostatic hyperplasia (BPH)).

The data also show that GCNT1, ST6GAL1 and FUT8 protein levels in blood or urine can be used to accurately distinguish between localised and metastatic prostate cancer, which cannot be done with PSA. This particular combination of three markers (GCNT1, ST6GAL1 and FUT8; referred to as “metastatic Glycoscore” herein) is therefore particularly beneficial when trying to distinguish between metastatic and localised prostate cancer.

As will be noted by a person of skill in the art, the term “glycoscore” is used herein to refer to a value that is obtained from a combination of at least two markers. Typically, but not exclusively, the term “glycoscore” is used herein to refer to the combination of GCNT1, GALNT7 and ST6GAL1; and the term “metastatic glycoscore” is typically but not exclusively used herein to refer to the combination of GCNT1, ST6GAL1 and FUT8.

The markers provided herein are also shown to be useful in distinguishing between prostate cancers of different Gleason score cytologies.

GCNT1, GALNT7, FUT8 and/or ST6GAL1 are therefore useful biomarkers for prostate cancer diagnosis, prognosis and patient stratification, without the need for tissue biopsy. The methods and markers described herein may therefore be used to reduce the large number of unnecessary tissue biopsies performed each year and identify patients with aggressive cancers that might have been missed by current PSA testing.

The markers described herein are typically detected in the biological fluid (e.g. blood or urine) at the protein level but they may also be detected by enzyme activity. They may be may be detected at the protein level using any suitable technique. For example, a sandwich ELISA test as described in the Examples section below may be used. Such methods can easily be implemented into a clinical workflow and can performed in a few hours using small quantities of amounts of bodily fluid (e.g. blood or urine). Measurement of protein levels is advantageous (over e.g. methods that measure RNA or DNA markers) as protein samples tend to be more robust, which makes handling easier.

The markers described herein have been detected in blood and urine. However, any other suitable biological fluids (into which the glycosylation enzymes GCNT1, GALNT7, FUT8 and/or ST6GAL1 may be secreted) may also be used. For example, a saliva sample, or a prostatic fluid sample (such as a prostatic secretion sample) may also be used.

In one aspect, the invention provides an in vitro method for diagnosing prostate cancer or determining the risk of developing prostate cancer in a subject, the method comprising the steps of:

a) determining the level of one or more biomarker in a biological fluid sample from the subject, wherein the one or more biomarker is selected from the group consisting of: GALNT7, ST6GAL1, FUT8 and GCNT1;
b) comparing the level of the one or more biomarker with the level of the same biomarker in a control sample or with a pre-determined reference level for the same biomarker; and
c) identifying a subject as having prostate cancer or as having an increased risk of developing prostate cancer if the comparison in step b) indicates that the subject has one or more of the following: an increased level of GALNT7 compared to the control sample or the pre-determined reference level; an increased level of ST6GAL1 compared to the control sample or the pre-determined reference level; a decreased level of GCNT1 compared to the control sample or the pre-determined reference level; an increased level of FUT8 compared to the control sample or the pre-determined reference level.

Suitably, the control sample may be from a control subject that does not have prostate cancer. Optionally the control sample may be from a subject that has benign prostatic hyperplasia, prostatitis or an enlarged prostate.

Suitably, the pre-determined reference level may be the average level of the biomarker in a control subject that does not have prostate cancer. Optionally the pre-determined reference level may be the average level of the biomarker in a subject that has benign prostatic hyperplasia, prostatitis or an enlarged prostate.

The inventors have shown that GCNT1 levels decrease in the blood and urine of subjects with prostate cancer when compared to subjects that do not have prostate cancer or have BPH, and that the levels of GCNT1 correlate with Gleason score. In addition, they have shown that for patients with prostate cancer, levels of GCNT1 increase for metastatic prostate cancer subjects compared to subjects with non-metastatic, localised, prostate cancer. GCNT1 levels can therefore be used in diagnosing prostate cancer or determining the risk of developing prostate cancer in a subject (diagnosed by a decrease compared to controls with no prostate cancer or BPH), and also in diagnosing metastatic prostate cancer or determining the risk of developing metastatic prostate cancer in a subject (diagnosed by an increase compared to localised, non-metastatic cancer). Diagnosing prostate cancer or metastatic prostate cancer can be followed by administering to the subject a treatment regimen suitable for the type of cancer.

In another aspect, the invention provides an in vitro method for diagnosing metastatic prostate cancer or determining the risk of developing metastatic prostate cancer in a subject, the method comprising the steps of:

a) determining the level of one or more biomarker in a biological fluid sample from the subject, wherein the one or more biomarker is selected from the group consisting of: ST6GAL1, FUT8, GCNT1 and GALNT7;
b) comparing the level of the one or more biomarker with the level of the same biomarker in a control sample or with a pre-determined reference level for the same biomarker, wherein the control sample is from a subject that has non-metastatic, localised, prostate cancer or the pre-determined reference level is the average level of the biomarker in a subject with non-metastatic, localised, prostate cancer; and
c) identifying a subject as having metastatic prostate cancer or as having an increased risk of developing metastatic prostate cancer if the comparison in step b) indicates that the subject has one or more of the following: an increased level of ST6GAL1 compared to the control sample or the pre-determined reference level; an increased level of GCNT1 compared to the control sample or the pre-determined reference level; an increased level of FUT8 compared to the control sample or the pre-determined reference value; or an increased level of GALNT7 compared to the control sample or the pre-determined reference level.

Suitably, the biological fluid sample may be blood or urine.

Suitably, step a) may comprise determining the level of at least two or three the recited biomarkers in the biological fluid sample.

Suitably, step a) may comprise determining the level of:

### ST6GAL1 and GALNT7;

### ST6GAL1 and GCNT1;

### ST6GAL1, GCNT1 and GALNT7;

### ST6GAL1, GCNT1 and FUT8; or

### ST6GAL1, GCNT1, GALNT7 and FUT8;

in the biological fluid sample.

Suitably, the subject may be human.

Suitably, the level of biomarker may be determined at the protein level, optionally using a process selected from: ELISA assay, immunoblotting, lateral flow assay, protein microarray and mass spectrometry.

Suitably, the method may further comprise selecting a treatment regimen for the subject based on the comparison of the level of the biomarker with the control sample or with the pre-determined reference level.

Suitably, the method may further comprise administering the selected treatment regimen to the subject, optionally wherein the selected treatment regimen comprises surgery, radiotherapy, chemotherapy, immunotherapy, hormone therapy, ultrasound therapy, or combinations thereof.

Suitably, the method may further comprise determining the level of PSA in the biological fluid sample.

In another aspect, the invention provides the use of one or more biomarkers selected from the group consisting of: GALNT7, ST6GAL1, FUT8 and GCNT1 as a biological fluid biomarker for prostate cancer.

Suitably, the use may be for distinguishing between non-metastatic, localised, prostate cancer and metastatic prostate cancer.

Suitably, the biomarkers may be:

### ST6GAL1 and GALNT7;

### ST6GAL1 and GCNT1;

### ST6GAL1, GCNT1 and GALNT7;

### ST6GAL1, GCNT1 and FUT8; or

### ST6GAL1, GCNT1, GALNT7 and FUT8.

Suitably, PSA may be used as an additional biomarker.

In another aspect, the invention provides an in vitro method for monitoring prostate cancer progression in a subject, the method comprising the steps of:

i) determining the level of one or more biomarker in a biological fluid sample from the subject in accordance with method steps a) to b) of the invention described above; and
ii) repeating step i) for the same subject after a time interval; and
iii) comparing the biomarker levels identified in i) with the biomarker levels identified in ii), wherein a change in the biomarker levels from i) to ii) is indicative of a change in prostate cancer progression in the subject.

Suitably, the method may be for monitoring for relapse into castrate resistant prostate cancer.

In another aspect, the invention provides an in vitro method for determining the therapeutic effect of a treatment regimen for prostate cancer, the method comprising:

a) determining the level of one or more biomarker in a biological fluid sample from the subject, wherein the one or more biomarker is selected from the group consisting of GALNT7, ST6GAL1, FUT8 and GCNT1;
b) repeating step a) using a biological fluid sample obtained from the subject after treatment for a time interval; and
c) comparing the level of biomarker determined in step a) to that determined in step b), and identifying that the treatment regimen has a therapeutic effect if one or more of the following is observed: there is a decrease in the level of GALNT7 after treatment; there is a decrease in the level of ST6GAL1 after treatment; there is a decrease in the level of FUT8 after treatment; or there is a change in the level of GCNT1 after treatment.

In one example, the change in level of GCNT1 that is indicative of a therapeutic effect is an increase in GCNT1 level after treatment.

As would be clear to a person of skill in the art, the direction of change in GCNT1 levels that is indicative of a therapeutic effect may depend on the disease status of the subject prior to treatment and the control/reference used. As a non-limiting example, if the subject has non-metastatic prostate cancer prior to treatment, an increase in GCNT1 levels (e.g. returning to levels equivalent to those observed in a subject with no prostate cancer or with BPH) may be indicative of a therapeutic effect. Other appropriate examples would be clear to a person of skill in the art, in the context of the invention disclosed herein.

In another aspect, the invention provides an in vitro method for determining a subject's compliance or adherence with a prescribed treatment regimen for prostate cancer, the method comprising:

a) determining the level of one or more biomarker in a biological fluid sample from the subject, wherein the one or more biomarker is selected from the group consisting of GALNT7, ST6GAL1, FUT8 and GCNT1;
b) repeating step a) after a time interval using a biological fluid sample from the subject after the prescribed start of treatment regimen; and
c) comparing the level of biomarker determined in step a) to that determined in step b), and identifying that the subject has complied or adhered with the prescribed treatment regimen if one or more of the following is observed: there is a decrease in the level of GALNT7 after treatment; there is a decrease in the level of ST6GAL1 after treatment; there is a decrease in the level of FUT8 after treatment; or there is a change in the level of GCNT1 after treatment.

In one example, the change in level of GCNT1 that is indicative of compliance or adherence with the prescribed treatment is an increase in GCNT1 level after treatment.

As would be clear to a person of skill in the art, the direction of change in GCNT1 levels that is indicative of compliance or adherence with the prescribed treatment may depend on the disease status of the subject prior to treatment and the control/reference used. As a non-limiting example, if the subject has non-metastatic prostate cancer prior to treatment, an increase in GCNT1 levels (e.g. returning to levels equivalent to those observed in a subject with no prostate cancer or with BPH) may be indicative of compliance or adherence with the prescribed treatment. Other appropriate examples would be clear to a person of skill in the art, in the context of the invention disclosed herein.

Suitably, the treatment may comprise surgery, radiotherapy, chemotherapy, immunotherapy, hormone therapy, ultrasound therapy, or combinations thereof.

Suitably, the biological fluid sample may be blood or urine.

Suitably, the level of at least two biomarkers selected from the group consisting of: GALNT7, ST6GAL1, FUT8 and GCNT1 may be determined in the biological fluid sample.

Suitably, the level of:

### ST6GAL1 and GALNT7;

### ST6GAL1 and GCNT1;

### ST6GAL1, GCNT1 and GALNT7;

### ST6GAL1, GCNT1 and FUT8; or

### ST6GAL1, GCNT1, GALNT7 and FUT8

may be determined in the biological fluid sample.

Suitably, the subject may be human.

Suitably, the level of biomarker may be determined at the protein level, optionally using a process selected from the group consisting of immunoblotting, lateral flow assay, ELISA assay, protein microarray and mass spectrometry.

Suitably, the method may further comprise determining the level of PSA in the biological fluid sample.

In another aspect, the invention provides a method of determining the clinical significance of prostate cancer in a subject, the method comprising:

determining the level of one or more biomarker in a biological fluid sample from the subject, wherein the one or more biomarker is selected from the group consisting of: GALNT7, ST6GAL1, FUT8 and GCNT1; and determining therefrom the clinical significance of the prostate cancer.

Suitably, the method may be for differentiating between subjects likely to exhibit normal prostate tissue or Gleason score <6 cytology, and those likely to have Gleason score >6 cytology.

Suitably, the method may be for differentiating between subjects likely to exhibit Gleason score cytology of less than or equal to 8, and those likely to have Gleason score cytology of more than or equal to 9.

Suitably, the method may be for diagnosing metastatic prostate cancer or determining the risk of developing metastatic prostate cancer.

Suitably, the method may comprise the step of selecting subjects to undergo further investigation and/or selecting subjects for prostate cancer treatment.

In another aspect, the invention provides a kit for diagnosing prostate cancer or determining the risk of developing prostate cancer in a subject, comprising:

(i) a detectably labelled agent that specifically binds to ST6GAL1 protein; and
(ii) one or more of:
a) a detectably labelled agent that specifically binds to GCNT1 protein;
b) a detectably labelled agent that specifically binds to GALNT7 protein; and
c) a detectably labelled agent that specifically binds to FUT8 protein.

Suitably, the kit may further comprise a detectably labelled agent that specifically binds to PSA protein.

Suitably, the kit may comprise one or more reagents for detecting the detectably labelled agent(s).

In a further aspect, the invention provides an assay device for diagnosing prostate cancer or determining the risk of developing prostate cancer in a subject, the device comprising a surface with at least two detectably labelled agents located thereon, wherein the at least two detectably labelled agents are:

(i) a detectably labelled agent that specifically binds to ST6GAL1 protein; and
(ii) one or more of:
a) a detectably labelled agent that specifically binds to GCNT1 protein;
b) a detectably labelled agent that specifically binds to GALNT7 protein; and
c) a detectably labelled agent that specifically binds to FUT8 protein.

Suitably, the device may further comprise a detectably labelled agent that specifically binds to PSA protein.

Suitably, the at least two detectably labeled agents may be located in separate zones on the surface.

In another aspect, the invention provides a method of diagnosing and treating prostate cancer in a subject, the method comprising the steps of:

a) measuring the level of one or more biomarker in a biological fluid sample from the subject, wherein the one or more biomarker is selected from the group consisting of: GALNT7, ST6GAL1, FUT8 and GCNT1;
b) comparing the level of the one or more biomarker with the level of the same biomarker in a control sample or with a pre-determined reference level for the same biomarker;
c) identifying a subject as having prostate cancer if the comparison in step b) indicates that the subject has one or more of the following: an increased level of GALNT7 compared to the control sample or the pre-determined reference level; an increased level of ST6GAL1 compared to the control sample or the pre-determined reference level; a decreased level of GCNT1 compared to the control sample or the pre-determined reference level; an increased level of FUT8 compared to the control sample or the pre-determined reference level; and
d) administering to the subject that has been identified as having prostate cancer a treatment for prostate cancer using therapy or surgery.

In another aspect, the invention provides a method of treating prostate cancer in a subject, the method comprising the steps of administering a prostate cancer treatment to a subject that has one or more of the following: an increased level of GALNT7 compared to a control sample or the pre-determined reference level; an increased level of ST6GAL1 compared to a control sample or the pre-determined reference level; a decreased level of GCNT1 compared to a control sample or the pre-determined reference level; an increased level of FUT8 compared to a control sample or the pre-determined reference level.

In another aspect, the invention provides a method of treating metastatic prostate cancer in a subject, the method comprising the steps of administering a metastatic prostate cancer treatment to a subject that has an increased level of FUT8 compared to a control sample or the pre-determined reference level and one or both of the following: an increased level of ST6GAL1 compared to a control sample or the pre-determined reference level; a decreased level of GCNT1 compared to a control sample or the pre-determined reference level.

Appropriate treatments are discussed elsewhere herein.

In another aspect, the invention provides a method for diagnosing and treating metastatic prostate cancer in a subject, the method comprising the steps of:

a) measuring the level of one or more biomarker in a biological fluid sample from the subject, wherein the one or more biomarker is selected from the group consisting of: ST6GAL1, FUT8, GCNT1 and GALNT7;
b) comparing the level of the one or more biomarker with the level of the same biomarker in a control sample or with a pre-determined reference level for the same biomarker, wherein the control sample is from a subject that has non-metastatic, localised, prostate cancer or the pre-determined reference level is the average level of the biomarker in a subject with non-metastatic, localised, prostate cancer;
c) identifying a subject as having metastatic prostate cancer if the comparison in step b) indicates that the subject has one or more of the following: an increased level of ST6GAL1 compared to the control sample or the pre-determined reference level; an increased level of GCNT1 compared to the control sample or the pre-determined reference level; an increased level of FUT8 compared to the control sample or the pre-determined reference value; or an increased level of GALNT7 compared to the control sample or the pre-determined reference level; and
d) treating the subject for prostate cancer using therapy or surgery.

In another aspect, the invention provides a method of treating a subject with prostate cancer (e.g. metastatic prostate cancer), the method comprising treating the subject by surgery or therapy, wherein the patient has been diagnosed as having prostate cancer (e.g. metastatic prostate cancer) using a method described elsewhere herein.

In another aspect, the invention provides a method of detecting prostate cancer in a subject, the method comprising:

measuring the level of one or more biomarker in a biological fluid sample from the subject, wherein the one or more biomarker is selected from the group consisting of: ST6GAL1, GCNT1, FUT8 and GALNT7, by contacting the biological fluid sample with an appropriate antibody specific to the biomarker of interest and detecting binding between the biomarker and the corresponding antibody (i.e. ST6GAL1 binding to an anti-ST6GAL1 antibody; GCNT1 binding to an anti-GCNT1 antibody, FUT8 binding to an anti-FUT8 antibody or GALNT7 binding to an anti-GALNT7 antibody).

In another aspect, the invention provides a method of detecting metastatic prostate cancer in a subject, the method comprising:

measuring the level of one or more biomarker in a biological fluid sample from the subject, wherein the one or more biomarker is selected from the group consisting of: ST6GAL1, FUT8, GCNT1 and GALNT7, by contacting the biological fluid sample with an appropriate antibody specific to the biomarker of interest and detecting binding between the biomarker and the corresponding antibody (i.e. ST6GAL1 binding to an anti-ST6GAL1 antibody; GCNT1 binding to an anti-GCNT1 antibody, FUT8 binding to an anti-FUT8 antibody; or GALNT7 binding to an anti-GALNT7 antibody).

In another aspect, the invention provides a method of monitoring prostate cancer progression in a subject and treating the subject, the method comprising the steps of:

i) measuring the level of one or more biomarker in a biological fluid sample from the subject in accordance with method steps a) to b) of the invention described above; and
ii) repeating step i) for the same subject after a time interval;
iii) comparing the biomarker levels identified in i) with the biomarker levels identified in ii), wherein a change in the biomarker levels from i) to ii) is indicative of a change in prostate cancer progression in the subject; and
iv) treating the subject for prostate cancer using therapy or surgery.

In another aspect, the invention provides a method of treating a subject with prostate cancer, the method comprising treating the subject by therapy or surgery, wherein the subject has been identified as having prostate cancer progression using a method described elsewhere herein.

In another aspect, the invention provides a method of monitoring prostate cancer progression in a subject, the method comprising:

i) measuring the level of one or more biomarker in a biological fluid sample from the subject, wherein the one or more biomarker is selected from the group consisting of: ST6GAL1, GCNT1, FUT8 and GALNT7, by contacting the biological fluid sample with an appropriate antibody specific to the biomarker of interest and detecting binding between the biomarker and the corresponding antibody (i.e. ST6GAL1 binding to an anti-ST6GAL1 antibody; GCNT1 binding to an anti-GCNT1 antibody, FUT8 binding to an anti-FUT8 antibody or GALNT7 binding to an anti-GALNT7 antibody);
ii) repeating step i) for the same subject after a time interval;
iii) comparing the biomarker levels identified in i) with the biomarker levels identified in ii), wherein a change in the biomarker levels from i) to ii) is indicative of a change in prostate cancer progression in the subject; and
iv) treating the subject for prostate cancer using therapy or surgery.

In another aspect, the invention provides a method for determining the therapeutic effect of a treatment regimen for prostate cancer, the method comprising:

a) measuring the level of one or more biomarker in a biological fluid sample from the subject, wherein the one or more biomarker is selected from the group consisting of GALNT7, ST6GAL1, FUT8 and GCNT1;
b) treating the subject according to a prescribed treatment regimen to the subject for a time interval;
c) repeating step a) using a biological fluid sample obtained from the subject after treatment for a time interval; and
c) comparing the level of biomarker determined in step a) to that determined in step b), and identifying that the treatment regimen has a therapeutic effect if one or more of the following is observed: there is a decrease in the level of GALNT7 after treatment; there is a decrease in the level of ST6GAL1 after treatment; there is a decrease in the level of FUT8 after treatment; or there is a change in the level of GCNT1 after treatment.

As stated in detail elsewhere herein, the direction of change in GCNT1 levels that is indicative of a therapeutic effect may depend on the disease status of the subject prior to treatment.

Returning the level of the biomarker to the levels comparable to the control in response to the treatment is indicative of remission.

The level of one or more biomarker in a biological fluid sample from the subject may be determined in any of the methods by contacting the biological fluid sample with an appropriate antibody specific to the biomarker of interest and detecting binding between the biomarker and the corresponding antibody (i.e. ST6GAL1 binding to an anti-ST6GAL1 antibody; GCNT1 binding to an anti-GCNT1 antibody; FUT8 binding to an anti-FUT8 antibody or GALNT7 binding to an anti-GALNT7 antibody).

In one aspect, a method is provided for determining the status of prostate tissue in a male subject, the method comprising measuring from a biological fluid of the subject the amount of a biomarker selected from GALNT7, ST6GAL1, FUT8 and GCNT1, wherein the amount of the biomarker measured in the biological fluid sample is indicative of the status of the prostate tissue. In this context, the term “status” includes healthy prostate tissue, or prostate tissue that is affected with cancer or metastatic cancer.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows that higher levels of GALNT7 and ST6GAL1 are present in the blood of men with prostate cancer compared to controls (A, B). ST6GAL1 can also be used to distinguish between non-metastatic (localised) prostate cancer and metastatic prostate cancer (C). The enzymes were detected using a simple sandwich ELISA assay. Note: data in (A) and (B) is generated from plasma samples; (C) is generated from serum samples.

FIG. 2 shows GALNT7 levels in urine samples from 180 men taking part in a clinical trial can distinguish between prostate cancer and non-cancer more accurately than PSA.

FIG. 3 shows a comparison of GALNT7 levels in plasma and urine samples from the same patients.

FIG. 4 shows results from a ST6GAL1 tissue microarray, wherein it is demonstrated that levels of ST6GAL1 protein are reduced in cancer tissue relative to benign tissue. ST6GAL1 was identified as upregulated at the RNA level in normal/benign tissue and prostate cancer (Munkley et al., 2016). Since then the inventors have studied ST6GAL1 in additional cohorts and found a lot of variation at the RNA level. Unexpectedly, as shown in FIG. 4, the level of protein was down in prostate cancer compared to benign prostatic hyperplasia. The inventors have extensively validated the antibody (FIG. 5 below) to show that it is specific. These data suggest that the levels of ST6GAL1 RNA/protein in tissue do not directly correlate with the levels of ST6GAL1 protein in patient blood.

FIG. 5 shows ST6GAL1 antibody validation for IHC (using an antibody previously validated and published by the inventors (Munkley et al., 2016) for use in western blot). A. shows IHC following pre-incubation of ST6GAL1 antibody with immunising peptide (1:6000 dilution); B. shows western blot data following pre-incubation of ST6GAL1 antibody with immunising peptide (1:1000 dilution); and C. shows IHC staining using FFPE fixed LNCaP stable cell lines (1:1000 dilution).

FIG. 6 shows that GALNT7 protein is upregulated in prostate cancer tissue in multiple patient cohorts.

FIG. 7 shows ELISA data which demonstrates that FUT8 is increased in the serum of men with metastatic prostate cancer relative to men with localised disease.

FIG. 8 shows that combining GCNT1, FUT8 and ST6GAL1 (to generate a metastatic GlycoScore) has the best power to distinguish localised and metastatic disease.

FIG. 9 shows RNA data from four patient cohorts wherein FUT8 is upregulated in prostate cancer. A. cohort 1 data shows that FUT8 is increased in a sub-group of primary prostate cancer with metastatic biology. B. cohort 2 data shows that FUT8 is increased metastatic prostate cancer relative to localised disease. C. Cohort 3 data shows that FUT8 is increased in prostate cancer relative to BPH. D. Cohort 4 data shows that FUT8 is increased in prostate cancer relative to BPH.

FIG. 10 shows that FUT8 protein levels in plasma increase in cancer relative to BPH (ELISA data from two different cohorts).

FIG. 11 shows that the combined analysis of three glycosylation enzymes (GlycoScore; in this case GCNT1, GALNT7 and ST6GAL1) in blood can distinguish between BPH and prostate cancer more accurately than PSA.

FIG. 12 shows that GALNT7 and ST6GAL1 in urine can both distinguish between benign and prostate cancer. The combined analysis of GALNT7 and ST6GAL1 in urine can distinguish between benign and prostate cancer more accurately than PSA. The data in FIG. 12 represents a cohort of 183 urine samples.

FIG. 13 shows that GALNT7 and ST6GAL1 are increased whilst GCNT1 is decreased in blood (serum) in prostate cancer relative to control, and that analysis of the combination (GALNT7, ST6GAL1 and GCNT1) can be used to distinguish between benign and prostate cancer.

FIG. 14 shows that analysis of a combination of GALNT7, ST6GAL1 and GCNT1 can be used to distinguish between benign and prostate cancer more accurately than PSA (based on 61 serum samples).

FIG. 15 shows that GALNT7, ST6GAL1 and GCNT1 can be used to distinguish between prostate cancers with different Gleason score cytologies (data generated using plasma samples).

FIG. 16 provides a summary of the treatment options for prostate cancer. The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

The inventors have surprisingly identified four new protein biomarkers, GCNT1, GALNT7, FUT8 and ST6GAL1 that are found in the biological fluids (e.g. blood or urine) of men with prostate cancer.

The biomarkers FUT8, GALNT7, ST6GAL1 and/or GCNT1 can be used for diagnosing prostate cancer or determining the risk of developing prostate cancer in a subject compared to a control (e.g. non-diseased patients, patients with BPH, or patients with enlarged prostate for example). ST6GAL1, GCNT1, GALNT7 and/or FUT8 have also been found to be particularly useful when distinguishing between localised prostate cancer and metastatic prostate cancer or determining the risk of developing metastatic prostate cancer in a subject. These markers can therefore be used to diagnose or determine the risk of developing prostate cancer per se, or more specifically be used to determine the type of prostate cancer (localised vs metastatic). One or more (e.g. two, three or four) of these biomarkers can advantageously be used in any of the methods, kits, assays, or uses described herein.

### Methods for Diagnosing Prostate Cancer or Determining the Risk of Developing Prostate Cancer in a Subject

In one aspect, an in vitro method for diagnosing prostate cancer or determining the risk of developing prostate cancer in a subject is provided, the method comprising the steps of:

a) determining the level of one or more biomarker in a biological fluid sample from the subject, wherein the one or more biomarker is selected from the group consisting of: GALNT7, ST6GAL1, FUT8 and GCNT1;
b) comparing the level of the one or more biomarker with the level of the same biomarker in a control sample or with a pre-determined reference level for the same biomarker; and
c) identifying a subject as having prostate cancer or as having an increased risk of developing prostate cancer if the comparison in step b) indicates that the subject has one or more of the following: an increased level of GALNT7 compared to the control sample or the pre-determined reference level; an increased level of ST6GAL1 compared to the control sample or the pre-determined reference level; an increased level of FUT8 compared to the control sample or the pre-determined reference level; a decreased level of GCNT1 compared to the control sample or the pre-determined reference level.

In a further aspect, an in vitro method for diagnosing metastatic prostate cancer or determining the risk of developing metastatic prostate cancer in a subject is provided, the method comprising the steps of:

a) determining the level of one or more biomarker in a biological fluid sample from the subject, wherein the one or more biomarker is selected from the group consisting of: ST6GAL1, FUT8, GCNT1 and GALNT7;
b) comparing the level of the one or more biomarker with the level of the same biomarker in a control sample or with a pre-determined reference level for the same biomarker, wherein the control sample is from a subject that has non-metastatic, localised, prostate cancer or the pre-determined reference level is the average level of the biomarker in a subject with non-metastatic, localised, prostate cancer; and
c) identifying a subject as having metastatic prostate cancer or as having an increased risk of developing metastatic prostate cancer if the comparison in step b) indicates that the subject has one or more of the following: an increased level of ST6GAL1 compared to the control sample or the pre-determined reference level; an increased level of GCNT1 compared to the control sample or the pre-determined reference level; an increased level of FUT8 compared to the control sample or the pre-determined reference value; or an increased level of GALNT7 compared to the control sample or the pre-determined reference level.

The term “subject” as used herein refers to, for example, humans, chimpanzees, Rhesus monkeys, dogs, cows, horses, cats, mice, rats, chickens, zebrafish, fruit flies, mosquitoes, C. elegans and frogs (all of which have one or more of GALNT7, ST6GAL1, FUT8 and GCNT1; or orthologues thereof), provided that they also have a prostate. The subject is preferably a mammal, such as a human. The subject is most commonly male.

The subject may be referred to herein as a patient. The terms “subject”, “individual”, and “patient” are used herein interchangeably. The subject can be symptomatic (e.g., the subject presents symptoms associated with prostate cancer), or the subject can be asymptomatic (e.g., the subject does not present symptoms associated with prostate cancer).

The subject may be diagnosed with, be at risk of developing or present with symptoms of prostate cancer. The subject may have, or be suspected of having (e.g. present with symptoms or a history indicative or suggestive of), prostate cancer.

Accordingly, in some examples, the subject has prostate cancer (and the method diagnoses, identifies, (or detects) that the subject has prostate cancer). In this context, the terms “diagnose” “identify”, and “detect” can be used interchangeably.

In particular examples, the subject has early stage prostate cancer. An example of an early stage of disease is when the subject has the initial symptoms of prostate cancer but has not yet developed sufficient symptoms for diagnosis of disease. in such examples, the method may be considered as a method for determining the risk of developing prostate cancer. In particular examples, the subject has localised prostate cancer. In other examples, the subject has metastatic prostate cancer.

The terms “cancer” and “cancerous” refer to or describe the physiological condition that is typically characterized by unregulated cell growth. Examples of cancer include cancer of the urogenital tract, such as prostate cancer. As used herein, the term “prostate cancer” refers to all stages and all forms of cancer arising from the tissue of the prostate gland.

Methods of diagnosing and staging prostate cancer are well known in the art. For example, according to the tumor, node, metastasis (TNM) staging system of the American Joint Committee on Cancer (AJCC), AJCC Cancer Staging Manual (7th Ed., 2010), the various stages of prostate cancer are defined as follows: Tumor: T1: clinically inapparent tumor not palpable or visible by imaging, T1a: tumor incidental histological finding in 5% or less of tissue resected, T1b: tumor incidental histological finding in more than 5% of tissue resected, T1c: tumor identified by needle biopsy; T2: tumor confined within prostate, T2a: tumor involves one half of one lobe or less, T2b: tumor involves more than half of one lobe, but not both lobes, T2c: tumor involves both lobes; T3: tumor extends through the prostatic capsule, T3a: extracapsular extension (unilateral or bilateral), T3b: tumor invades seminal vesicle(s); T4: tumor is fixed or invades adjacent structures other than seminal vesicles (bladder neck, external sphincter, rectum, levator muscles, or pelvic wall). Node: N0: no regional lymph node metastasis; N1: metastasis in regional lymph nodes. Metastasis: M0: no distant metastasis; M1: distant metastasis present.

The Gleason Grading system is also commonly used to help evaluate the prognosis of men with prostate cancer. Together with other parameters, it is incorporated into a strategy of prostate cancer staging, which predicts prognosis and helps guide therapy. A Gleason “score” or “grade” is given to prostate cancer based upon its microscopic appearance. Tumors with a low Gleason score typically grow slowly enough that they may not pose a significant threat to the patients in their lifetimes. These patients are monitored (“watchful waiting” or “active surveillance”) over time. Cancers with a higher Gleason score are more aggressive and have a worse prognosis, and these patients are generally treated with surgery (e.g., radical prostectomy) and, in some cases, therapy (e.g., radiation, hormone, ultrasound, chemotherapy, immunotherapy). Gleason scores (or sums) comprise grades of the two most common tumor patterns. These patterns are referred to as Gleason patterns 1-5, with pattern 1 being the most well-differentiated. Most have a mixture of patterns. To obtain a Gleason score or grade, the dominant pattern is added to the second most prevalent pattern to obtain a number between 2 and 10. The Gleason Grades include: G1: well differentiated (slight anaplasia) (Gleason 2-4); G2: moderately differentiated (moderate anaplasia) (Gleason 5-6); G3-4: poorly differentiated/undifferentiated (marked anaplasia) (Gleason 7-10).

The methods described herein may be used to identify subjects that have prostate cancer or that have an increased risk of developing prostate cancer. In this context, the phrase “increased risk” indicates that the subject has a higher level of risk (or likelihood) that they will experience a particular clinical outcome. A subject may be classified into a risk group or classified at a level of risk based on the methods described herein, e.g. high, medium, or low risk. A “risk group” is a group of subjects or individuals with a similar level of risk for a particular clinical outcome.

In general, the methods described are in vitro methods that are performed using a sample that has already been obtained from the subject (i.e. the sample is provided for the method, and the steps taken to obtain the sample from the subject are not included as part of the method).

The methods may therefore include the step of providing a biological fluid sample from a subject.

As used herein, “provide”, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample).

The methods provided herein comprise providing a biological fluid sample (for example a blood sample or a urine sample) from a subject. The samples being tested in the methods described herein are also referred to as “test samples”.

As used herein, the terms “biological sample”, “test sample”, “sample” and variations thereof refer to a sample obtained or derived from a subject. For the purposes described herein, the sample is, or comprises, a biological fluid (also referred to herein as a bodily fluid) sample.

As used herein, the term “biological fluid sample” encompasses a blood sample, a urine sample, a saliva sample, or a prostatic fluid sample (such as a prostatic secretion sample).

A blood sample may be a whole blood sample, or a processed blood sample e.g. serum, plasma etc. Methods for obtaining biological fluid samples (e.g. whole blood, serum, plasma, urine etc) from a subject are well known in the art. For example, methods for obtaining blood samples from a subject are well known and include established techniques used in phlebotomy. The obtained blood samples may be further processed using standard techniques to obtain e.g. a serum sample, or a plasma sample. Advantageously, methods for obtaining biological fluid samples from a subject are typically low-invasive or non-invasive.

A whole blood sample is defined as a blood sample drawn from the human body and from which (substantially) no constituents (such as platelets or plasma) have been removed. In other words, the relative ratio of constituents in a whole blood sample is substantially the same as a blood in the body. In this context, “substantially the same” allows for a very small change in the relative ratio of the constituents of whole blood e.g. a change of up to 5%, up to 4%, up to 3%, up to 2%, up to 1% etc. Whole blood contains both the cell and fluid portions of blood. A whole blood sample may therefore also be defined as a blood sample with (substantially) all of its cellular components in plasma, wherein the cellular components (i.e. at least comprising the requisite white blood cells, red blood cells, platelets of blood) are intact.

In a preferred example, the biological fluid sample is plasma, serum or urine.

Methods for analysing (and optionally isolating, enriching for or extracting) protein biomarkers from blood, plasma, serum, saliva, prostatic fluid and urine samples have been described previously, see for example; Heitzer, E., Haque, I. S., Roberts, C. E. S. et al. Current and future perspectives of liquid biopsies in genomics-driven oncology. Nat Rev Genet 20, 71-88 (2019).

The methods provided herein include the step of determining the level of one or more biomarker in a biological fluid sample from the subject, wherein the one or more biomarker is selected from the group consisting of: GALNT7, ST6GAL1, FUT8 and GCNT1.

A biomarker is an organic biomolecule (e.g. a protein, polypeptide, peptide, isomeric form thereof, immunologically detectable fragment thereof, corresponding nucleic acid molecule (e.g. mRNA, cDNA etc)) which is differentially present in a sample taken from a subject having a disease as compared with a subject not having the disease. A biomarker is differentially present if the mean or median level of the biomarker in the different groups is calculated to be statistically significant. Common tests for statistical significance include, among others, t-test (e.g., student t-test), ANOVA, Kruskal-Wallis, Wilcoxon, Mann-Whitney, Receiver Operating Characteristic (ROC curve), accuracy and odds ratio. Biomarkers, alone or in combination, provide measures of relative risk that a subject belongs to one phenotypic status or another. Therefore, they are useful as markers for disease (diagnostics), therapeutic effectiveness of a drug and drug toxicity.

Typically, the biomarker referred to herein is measured at the protein level.

“GALNT7 is also known as N-acetylgalactosaminyltransferase 7, polypeptide N-acetylgalactosaminyltransferase 7, UDP-GalNAc: polypeptide N-acetylgalactosaminyltransferase 7, UDP-N-acetyl-alpha-D-galactosamine:polypeptide N-acetylgalactosaminyltransferase 7 (GalNAc-T7), polypeptide GalNAc transferase 7 pp-GaNTase 7, protein-UDP acetylgalactosaminyltransferase 7, GalNAcT7, GALNAC-T7, NP_059119.2 (EC 2.4.1.41), XP_005263119.1 (EC 2.4.1.41), XP_011530327.1 (EC 2.4.1.41), XP_016863781.1 (EC 2.4.1.41), and XP_016863782.1 (EC 2.4.1.41)).

Initiation of O-glycosylation is carried out by a family of N-acetylgalactosamine (GalNAc)-transferase enzymes (including GALNT7) that catalyse the transfer of GalNAc to serine and threonine residues on target proteins to initiate O-linked glycosylation and produce the Tn antigen. The O-GalNAc residues are further processed by the addition of different monosaccharides.

GALNT7 may be human GALNT7. Human GALNT7 can be identified using NCBI GenBank or UniProt (NCBI Gene ID: NM_017423.2; NCBI Protein ID: NP_059119.2).

“ST6GAL1” (also known as beta-galactoside alpha-2,6-sialyltransferase 1, α-2,6-sialyltransferase, B-cell antigen CD75, CMP-N-acetylneuraminate beta-galactosamide alpha-2,6-sialyltransferase, CMP-N-acetylneuraminate-beta-galactosamide-alpha-2,6-sialyltransferase 1, ST6 N-acetylgalactosaminide alpha-2,6-sialyltransferase 1, ST6 beta-galactosamide alpha-2,6-sialyltranferase 1, ST6Gal I, alpha 2,6-ST 1, sialyltransferase 1 (beta-galactoside alpha-2,6-sialyltransferase), ST6N, SIAT1, ST6GalI NP_001340845.1 (EC 2.4.99.1), NP_003023.1 (EC 2.4.99.1), NP_775323.1 (EC 2.4.99.1), NP_775324.1 (EC 2.4.99.1)) is a glycosylation enzyme and Golgi type II membrane protein. ST6GAL1 catalyzes attachment of a 2,6 sialic acid to galactose on N-linked glycans.

The trans-Golgi enzyme ST6GAL1 has a cytoplasmic domain (cyto), a transmembrane domain (TMD), and an enzymatic luminal domain (lumen). The single hydrophobic segment serves as a signal-anchor sequence. This TMD spans the lipid bilayers of the secretory pathway, including the membrane of the Golgi apparatus. The enzymatic luminal domain of a glycosyltransferase is located within the lumen of the Golgi apparatus. The catalytic domain is located within the enzymatic luminal domain. The enzymatic transferase activity is located in the C-terminus of the protein. Membrane-tethered transferases are susceptible to proteolytic cleavages within its “stem” region. Proteolysis liberates a catalytically active, soluble form of the enzyme that may be released from the cell. ST6GAL1 has a b-secretase (BACE1) cleavage site in its luminal domain at EFQ41-43, which can result in its secretion.

Sialylation by ST6GAL1 typically occurs in the trans-Golgi where the glycosyl transferase is anchored by a transmembrane domain. Attachment of sialic acid to galactose on IgG Fc glycans is catalyzed by the ST6GAL1. Sialylation of IgG by ST6GAL1 typically occurs in the /ra//.s-Golgi where ST6GAL1 is found anchored in the Golgi by a transmembrane domain. This trans-Golgi enzyme can attach terminal sialic acid to complex biantennary glycans.

Several distinct promoters regulate the cellular and tissue specific expression of this transferase. For example, promoter 1 is used exclusively to express ST6GAL1 by hepatocytes, while B cells use promoter 2. Hepatocytes are responsible exclusively for production of soluble ST6GAL1 (sST6GAL1), which is cleaved and secreted into the circulation.

Human ST6GAL1 can be identified using NCBI GenBank or UniProt. Three different human isoforms have been identified. The corresponding NCBI references are:

1) NCBI Gene ID: NM_173217.2; NCBI protein ID: NP_775324.1;

### 2) NCBI Gene ID: NM_173216.2; NCBI Protein ID: NP_775323.1; and

### 3) NCBI Gene ID: NM_003032.2; NCBI Protein ID: NP_003023.1.

“GCNT1” (also known as glucosaminyl (N-acetyl) transferase 1, beta-1,3-galactosyl-O-glycosyl-glycoprotein beta-1,6-N-acetylglucosaminyltransferase, beta-1,6-N-acetylglucosaminyltransferase, core 2 GnT, core 2 beta-1,6-N-acetylglucosaminyltransferase I, core 2 beta1,6 N-acetylglucosaminyltransferase-I, core 2 branching enzyme, core2-GlcNAc-transferase, glucosaminyl (N-acetyl) transferase 1, core 2 (beta-1,6-N-acetylglucosaminyltransferase), NP_001091102.1 (EC 2.4.1.102), NP_001091103.1 (EC 2.4.1.102), NP_001091104.1 (EC 2.4.1.102), NP_001091105.1 (EC 2.4.1.102), NP_001481.2 (EC 2.4.1.102), G6NT, C2GNT, C2GNT1, NACGT2, NAGCT2, C2GNT-L) is located on chromosome 9ql3 and contains 1 exon that encodes a 428 amino acid protein. GCNT1 catalyzes the formation of core 2 O-glycans. Various physiological and pathological phenomena such as human T-cell activation, cell differentiation, immunodeficiency due to the Wiskott-Aldrich syndrome, leukemia and malignant transformation have been associated with changes in the core 2 structure or GCNT1 activity. GCNT1 may be human GCNT1. Human GCNT1 can be identified using NCBI GenBank or UniProt. Several different splice isoforms for GCNT1 are known, wherein the variation is in the untranslated region (resulting in the same protein). NCBI Protein IDs for exemplary GCNT1 isoforms include NP_001091103.1, NP_001091102.1, NP_001481.2, NP_001091104.1 and NP_001091105.1.

“FUT8” (also known as alpha-(1,6)-fucosyltransferase, GDP-L-Fuc:N-acetyl-beta-D-glucosaminide alpha1,6-fucosyltransferase, GDP-fucose-glycoprotein fucosyltransferase alpha1-6FucT, fucosyltransferase 8 (alpha (1,6) fucosyltransferase), glycoprotein 6-alpha-L-fucosyltransferase, CDGF, CDGF1, NP_001358462.1 (EC 2.4.1.68), NP_001358463.1 (EC 2.4.1.68), NP_001358465.1 (EC 2.4.1.68), NP_004471.4 (EC 2.4.1.68), NP_835368.1 (EC 2.4.1.68), NP_835369.1 (EC 2.4.1.68), XP_011534916.1 (EC 2.4.1.68), XP_016876625.1 (EC 2.4.1.68), XP_016876627.1 (EC 2.4.1.68), XP_016876628.1 (EC 2.4.1.68), XP_016876629.1 (EC 2.4.1.68)) is an enzyme responsible for transferring a fucose moiety from GDP-fucose to N-acetylglucosamine of N-glycan chain in proteins. Disruption of FUT8 function through various means leads to production of non-fucosylated proteins, including antibodies.

In mammalian expression systems, GDP-fucose, an essential substrate of fucosylation, is synthesized in the cytoplasm through de novo and salvage pathways. In the de novo pathway of fucosylation, GDP-fucose is synthesized through conversion of GDP-mannose to GDP-4-keto-6-deoxy-mannose, catalyzed by the enzyme GDP-mannose 4,6-dehydratase (GMD).

This GDP-Fucose is then transported inside the golgi and used as a substrate for protein fucosylation by the enzyme alpha-6 fucosyltransferase (FUT8). The enzyme transfers the fucose moiety from GDP-fucose to N-acetylglucosamine of the N-glycan chain. These critical enzymes, GDP-mannose 4,6-dehydratase and α,1-6 fucosyltransferase are encoded by GMD and FUT8 genes respectively.

FUT8 enzyme functions downstream of GDP-Fucose biosynthesis step and is the last enzymatic step for fucosylation of cellular proteins in golgi. Fucosylation precursors from both de novo and salvage pathway use FUT8 enzyme for final fucose moiety transfer. It is known that the α1-6 fucosyltransferase is the only enzyme responsible for adding fucose to the N-linked biantennary carbohydrate at Asn297 in the CH2 domain of the IgG antibody. Fucose attachment to the Fc core region is via an α-1,6 linkage generated by the FUT8 protein.

FUT8 may be human FUT8. Human FUT8 can be identified using NCBI GenBank or UniProt. Several different splice isoforms for FUT8 are known. NCBI Protein IDs for exemplary FUT8 isoforms include: NP_835369.1, NP_004471.4 and NP_835368.1.

“PSA” (also known as kallikrein related peptidase 3, APS, prostate-specific antigen (PSA), hK3, KLK2A1, P-30 antigen, gamma-seminoprotein, kallikrein-3, semenogelase, seminin NP_001025218.1 (EC 3.4.21.77), NP_001025219.1 (EC 3.4.21.77), NP_001639.1 (EC 3.4.21.77)) is a protein produced by the cells of prostate gland. Prostate Specific Antigen is a protein that is secreted by the epithelial cells of the prostate gland, including cancer cells.

Prostate-specific antigen (PSA) is a glycoprotein enzyme encoded by the KLK3 gene. KLK3 is a member of the kallikrein-related peptidase family and is secreted by the epithelial cells of the prostate gland. PSA is a kallikrein-like serine protease that is produced exclusively by the columnar epithelial cells lining the acini and ducts of the prostate gland. PSA mRNA is translated as an inactive 261-amino acid preproPSA precursor. PreproPSA has 24 additional residues that constitute the pre-region (the signal polypeptide) and the propolypeptide. Release of the propolypeptide results in the 237- amino acid, mature extracellular form, which is enzymatically active. PSA is organ-specific and, as a result, it is produced by the epithelial cells of benign prostatic hyperplastic (BPH) tissue, primary prostate cancer tissue, and metastatic prostate cancer tissue.

PSA is concentrated in prostatic tissue, and serum PSA levels are normally very low. Disruption of the normal prostate architecture, for example by prostatic disease, inflammation or trauma, allows greater amounts of PSA to enter the circulation. PSA is used to detect potential problems in the prostate gland and to follow the progress of prostate cancer therapy. PSA is not a specific marker for prostate cancer, since its levels increase due to other conditions, including prostatic hyperplasia, and PSA levels are also known to be affected by factors such as medication, urologic manipulation and inflammation. PSA is present in small quantities in serum of men with a healthy prostate, but is often elevated in individuals with prostate cancer and other prostate disorders. It has been established that between 40 and 45% of the variability in PSA levels in the general population is due to inherited factors.

A blood test to measure PSA is considered the most effective test currently available for the early detection of prostate cancer, although but its clinical effectiveness has been questioned.

Rising levels of PSA over time are associated with both localized and metastatic prostate cancer. In general, PSA values ranging from 2.5 ng/mL to 4 ng/mL are considered as cut-off values for suspected cancer, and levels above 10 ng/mL indicate higher risk. However, despite the widespread use of the PSA screening test, it is limited both in specificity and sensitivity and substantial controversy exists about its beneficial effect for patients. This is mainly due to the fact that PSA is not a specific marker of prostate cancer since its serum levels increase in prostatic hyperplasia and are affected by many other factors such as medication, urologic manipulations and inflammation. The probability that a patient with a positive (abnormal) PSA test result actually has prostate cancer is therefore relatively low, resulting in PSA testing in prostate cancer having a relatively low positive predictive value (PPV).

The decision to proceed with prostate biopsy is usually made based on results of a PSA assay, which is sometimes also followed by a Digital Rectal Examination (DRE). Results of PSA assay, alone or in combination with results of DRE, are used to select those individuals for prostate biopsy. Further factors may be considered, including free and total PSA, age of the patient, the rate of PSA change with age (PSA velocity), family history, ethnicity, history of prior biopsy etc.

PSA may be human PSA. Human PSA can be identified using NCBI GenBank or UniProt. Several different splice isoforms for PSA are known. NCBI Protein IDs for exemplary PSA isoforms include NP_001025219.1, NP_001025218.1 and NP_001639.1.

The methods provided herein refer to “determining” the level of one or more proteins. As would be clear to a person of skill in the art, the level of one or more proteins is typically “determined” by measuring the level of the protein in the sample. The term “determining” can therefore be replaced with the term “measuring” or “determining by measuring” herein.

Conventional “determining” methods may include sending a clinical sample(s) to a commercial laboratory for measurement of the biomarker levels in the biological fluid sample, or the use of commercially available assay kits for measuring the biomarker levels in the biological fluid sample. Exemplary kits and suppliers will be apparent to a person of skill in the art. In various examples, biomarkers may be determined, detected and/or quantified using ELISA assays or lateral flow devices, such as for point-of-care use, as well as spot check colorimetric tests.

The level of biomarker present in the biological fluid sample may be determined by e.g. assaying the amount of protein biomarker present in the sample. Assays for measuring the amount of a specified protein are well known in the art and include direct or indirect measures.

The level of protein biomarker in a sample may also be determined by determining the level of protein biomarker activity in a sample. Accordingly, protein “level” encompasses both the amount of protein per se, or its level of activity.

By way of example, the level of a protein biomarker in a biological fluid sample can be determined (e.g., measured) by any suitable methods and materials known in the art, including, for example, a process selected from the group consisting of mass spectrometry, immunoassays, enzymatic assays, spectrophotometry, colorimetry, fluorometry, bacterial assays, protein microarrays, compound separation techniques, or other known techniques for determining the presence and/or quantity of an analyte. Examples of relevant techniques include enzyme linked immunosorbent assays (ELISAs), immunoprecipitation, immunofluorescence, enzyme immunoassay (EIA), radioimmunoassay (RIA), Western blot analysis, and Lateral Flow (using e.g. Lateral Flow Devices (LFDs) utilizing a membrane bound antibody specific to the protein biomarker). Preferably, the level of a protein biomarker in a biological fluid sample is measured by ELISA or lateral flow.

In an example, the methods described herein determine the level of two or three or four of the specified biomarkers.

For example, the method may determine the level of GALNT7 and ST6GAL1; GALNT7 and GCNT1; or GALNT7 and FUT8.

The method may determine the level of GALNT7, ST6GAL1 and GCNT1. Alternatively, the method may determine the level of GALNT7, ST6GAL1 and FUT8. Alternatively, the method may determine the level of GALNT7, GCNT1 and FUT8.

In a further example, the method may determine the level of ST6GAL1 and GCNT1; or ST6GAL1 and FUT8. The method may also determine the level of ST6GAL1, GCNT1 and FUT8.

In a further example, the method may determine the level of GCNT1 and FUT8.

Alternatively, the method may determine the level of GALNT7, ST6GAL1, GCNT1 and FUT8.

In a preferred example, the methods may determine the level of at least GALNT7 and ST6GAL1 when the methods are for diagnosing prostate cancer or the risk of developing prostate cancer generally, or for monitoring prostate cancer progression (including prostate cancer relapse). In a further preferred example, the methods may determine the level of GALNT7, ST6GAL1 and GCNT1 (which may be referred to as Glycoscore herein) when the methods are for diagnosing prostate cancer or the risk of developing prostate cancer generally, or for monitoring prostate cancer progression (including prostate cancer relapse). In a further preferred example, methods for determining the therapeutic effect of appropriate treatment regimens for prostate cancer or determining a subject's compliance or adherence with a prescribed treatment regimen for prostate cancer generally may also determine the level of GALNT7, ST6GAL1 and GCNT1. In this context “prostate cancer generally” refers to all forms of prostate cancer, including but not limited to localised prostate cancer and metastatic prostate cancer.

In an alternative preferred example, the methods may determine the level of FUT8, ST6GAL1 and GCNT1 (which may be referred to as metastatic Glycoscore) when the methods are for diagnosing metastic prostate cancer or the risk of developing metastatic prostate cancer, or for monitoring metastatic prostate cancer progression (including metastatic prostate cancer relapse). In a further preferred example, methods for determining the therapeutic effect of appropriate treatment regimens for metastatic prostate cancer or determining a subject's compliance or adherence with a prescribed treatment regimen for metastatic prostate cancer may also determine the level of FUT8, ST6GAL1 and GCNT1.

Any of the above combinations may also be combined with PSA.

Methods described herein further comprise comparing the level of the at least one biomarker (i.e. its amount per se or its activity) in the biological fluid sample (“test sample”) with the level of the same biomarker in a control sample or with a predetermined reference level for the same biomarker.

In one example, the methods described may include contacting a control biological fluid sample with a compound or agent capable of detecting a specific biomarker protein (e.g. GALNT7 protein, ST6GAL1 protein, FUT8 protein or GCNT1 protein), and comparing the level of the biomarker protein in the control sample with the presence of the biomarker protein in the test sample.

As used herein “control sample”, refers to a sample having a normal level of biomarker (e.g. GCNT1, GALNT7, FUT8 and ST6GAL1), for example a sample obtained from at least one individual that does not have prostate cancer from the same species, or a sample obtained from at least one individual having benign prostatic hyperplasia from the same species. The individual can be the same age, sex or in the same state or condition of health as the subject from which the test sample is obtained.

As used herein, an individual that “does not have prostate cancer” is an individual that has histologically normal-appearing prostate tissue. Methods for histologically testing prostate tissue and identifying whether an individual has histologically normal-appearing prostate tissue are well known in the art, see for example Litwin M S and Tan H J., The Diagnosis and Treatment of Prostate Cancer: A Review. JAMA. 2017 Jun. 27; 317(24):2532-254. A control sample that is obtained from an individual that does not have prostate cancer in this context therefore refers to a biological fluid sample (e.g. a blood or urine sample, as appropriate) that has been obtained from an individual of the same species, where the individual has histologically normal-appearing prostate tissue. Examples of individuals that do not have prostate cancer include individuals with benign prostate hyperplasia, prostatitis and/or an enlarged prostate.

Benign prostate hyperplasia (BPH; also known as benign prostate enlargement) is a medical condition that is common in men aged over 50. It is a condition that can affect how urine is passed but is not a cancer and does not result in an increased risk of developing prostate cancer.

Accordingly, as used herein, an individual that has “benign prostatic hyperplasia” is an individual that has an enlarged prostate with histologically normal-appearing prostate tissue. Methods for histologically testing prostate tissue and identifying whether an individual has benign prostatic hyperplasia are well known in the art, see for example Chughtai et al. Benign prostatic hyperplasia. Nat Rev Dis Primers. 2016 May 5; 2:16031. A control sample that is obtained from an individual that has benign prostatic hyperplasia in this context therefore refers to a biological fluid sample (e.g. a blood or urine sample, as appropriate) that has been obtained from an individual of the same species, where the individual has an enlarged prostate with histologically normal-appearing prostate tissue.

Prostatitis is the name given to a set of symptoms which are thought to be caused by an infection or by inflammation of the prostate gland. Prostatitis is not a form of prostate cancer. It's a common condition which can affect men of any age, but it's most common in younger and middle aged men, typically between 30 and 50. Prostatitis can cause a wide range of symptoms, which vary from man to man. Common symptoms include problems passing urine and pain or discomfort around the testicles, back passage or lower abdomen. There are four types of prostatitis, Chronic pelvic pain syndrome (CPPS), Acute bacterial prostatitis, Chronic bacterial prostatitis and Asymptomatic prostatitis.

A control sample that is obtained from an “individual with prostatitis” therefore refers to a biological fluid sample (e.g. a blood or urine sample, as appropriate) that has been obtained from an individual of the same species, where the individual has been diagnosed with one of the above forms of prostatitis.

An enlarged prostate is an increase in the size of the prostate that is not caused by cancer. The medical term for an enlarged prostate is benign prostatic enlargement (BPE). It is also referred to as benign prostatic hyperplasia (BPH), which is described in more detail above.

The control sample may be assayed at the same time, before or after, separately or simultaneously with the test sample. The control value that is used in the comparison with the test sample may be a value that is calculated as an average or median of more than one (e.g. two or more, five or more, ten or more, a group etc) of control samples. Alternatively, the control sample may be a sample that originated from (i.e. is a mix of) more than one (e.g. two or more, five or more, ten or more, a group etc) individual that is not suffering from prostate cancer (or that has benign prostatic hyperplasia).

In one example, the control sample is therefore obtained from a control subject that does not have prostate cancer. In a further example, the control sample is obtained from a subject that has benign prostatic hyperplasia.

Alternatively, the level of biomarker (e.g. protein) in the biological fluid sample may be compared to a pre-determined reference level for the biomarker of interest.

As used herein, a “predetermined reference level” refers to a biomarker level obtained from a reference database, which may be used to generate a pre-determined cut off value, i.e. a score that is statistically predictive of prostate cancer. In one example, the predetermined reference level is the average or median level of the biomarker in at least one individual not suffering from prostate cancer from the same species. The predetermined reference value may be calculated as the average or median, taken from a group or population of individuals that are not suffering from prostate cancer. For example, the predetermined reference value may be calculated as the average or median, taken from a group or population of individuals that have benign prostatic hyperplasia. The individual or the population of individuals can be the same age, sex or in the same state or condition of health as the subject from which the test sample is obtained.

In one example, the pre-determined reference level is therefore the average level of the biomarker in a control subject that does not have prostate cancer. In a further example the pre-determined reference level is the average level of the biomarker in a subject that has benign prostatic hyperplasia.

Typically, in methods for diagnosing prostate cancer or determining the risk of developing prostate cancer in a subject, the control sample or predetermined reference are obtained from an individual or group of individuals that are distinct from the subject that is being tested (i.e. the subject from which the test sample is obtained/provided). In such examples, the control or predetermined reference are used as a bench line to determine whether the tested subject has or is at risk of having prostate cancer.

In an alternative example, the control or predetermined reference value may be obtained from the same individual as the test sample, but at an earlier time point. This is particularly relevant for the methods described herein that monitor prostate cancer progression in a subject, determine the therapeutic effect of a treatment regimen for prostate cancer, and/or determine a subject's compliance or adherence with a prescribed treatment regimen for prostate cancer. In such examples, the control sample or predetermined reference level is used to determine any changes in the level of the biomarker(s) over a time interval for the same subject.

The pre-determined reference level or control sample can therefore be from the same subject that the test sample is obtained from, for example obtained at an earlier time point. This earlier time point can be before they were diagnosed with or known to be at risk of developing prostate cancer.

A pre-determined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of a biomarker of a population of individuals comprising the subject to be investigated. How to calculate a suitable reference value, preferably, the average or median, is well known in the art. The population of subjects referred to before shall comprise a plurality of individuals, preferably, at least 5, 10, 50, 100, 1,000 subjects.

Thus, in some cases the level of the protein biomarker in a subject being greater than or equal to the level of the biomarker of the control sample or pre-determined reference level is indicative of a clinical status (e.g., indicative of prostate cancer). In other cases the level of the biomarker in a subject being less than or equal to the level of biomarker of the control sample or predetermined reference level is indicative of a clinical status (e.g. indicative of a therapeutic improvement or reversal in prostate cancer staging). The amount of the greater than and the amount of the less than is usually of a sufficient magnitude to, for example, facilitate distinguishing a subject from a control subject using the methods described herein. Typically, the greater than, or the less than, that is sufficient to distinguish a subject from a control subject is a statistically significant greater than, or a statistically significant less than. In cases where the level of the biomarker in a subject being equal to the level of the biomarker in a control subject is indicative of a clinical status, the “being equal” refers to being approximately equal (e.g., not statistically different).

The pre-determined value can depend upon a particular population of subjects (e.g., human subjects) selected. For example, an apparently healthy population will have a different ‘normal’ range of the protein biomarker than will a population of subjects which have, or are likely to have, prostate cancer. Accordingly, the pre-determined values selected may take into account the category (e.g., healthy, at risk, diseased) in which a subject (e.g., human subject) falls. Appropriate ranges and categories can be selected with no more than routine experimentation by those of ordinary skill in the art.

Suitably, the level of the specific biomarker detected in a sample (e.g. a test sample, a control sample etc) may be normalized by adjusting the measured level (amount or activity) of the biomarker using the level of a reference protein in the same sample, wherein the reference protein is not a marker itself (it is e.g., a protein that is constitutively expressed). This normalization allows the comparison of the biomarker level in one sample to another sample, or between samples from different sources. This normalized level can then optionally be compared to a reference value or control.

For example, when measuring a protein biomarker in a whole blood sample the biomarker may be expressed as an absolute concentration or, alternatively, it may be normalized against a known protein constitutively expressed in whole blood such as albumin, immunoglobulins or plasma protein concentration.

For example, when measuring a protein biomarker in a urine sample the biomarker may be expressed as an absolute concentration or, alternatively, it may be normalized against a known protein constitutively expressed in urine such as creatinine protein concentration.

For example, when measuring a protein biomarker in a serum (or plasma) sample the biomarker may be expressed as an absolute concentration or, alternatively, it may be normalized against a known protein constitutively expressed in serum (or plasma).

The biomarker level(s) in the test sample may be compared to the level of the same biomarker in a control sample or with a pre-determined reference level for the same biomarker to identify an increase or decrease in a level of the one or more biomarker in the sample of the subject. In the methods described herein, the subject may be identified as having prostate cancer or as having an increased risk of developing prostate cancer if the comparison (between biomarker level(s) in the control sample/predetermined reference value and the test sample of the subject) indicates that the subject has one or more of the following: an increased level of GALNT7 compared to the control sample or the pre-determined reference level; an increased level of ST6GAL1 compared to the control sample or the pre-determined reference level; an increased level of FUT8 compared to the control sample or the pre-determined reference level; or a decreased level of GCNT1 compared to the control sample or the pre-determined reference level.

In a particular example, the subject may be identified as having prostate cancer or as having an increased risk of developing prostate cancer when they have an increased level of GALNT7 in their blood or urine sample compared to the control sample or the pre-determined reference level (e.g. when the level of GALNT7 protein in their blood or urine sample is higher than the level of GALNT7 protein in the control sample or predetermined reference sample that has been obtained from an individual or individuals without prostate cancer or with benign prostatic hyperplasia).

In another particular example, the subject may be identified as having prostate cancer or as having an increased risk of developing prostate cancer when they have an increased level of ST6GAL1 in their blood or urine sample compared to the control sample or the pre-determined reference level (e.g. when the level of ST6GAL1 protein in their blood or urine sample is higher than the level of ST6GAL1 protein in the control sample or predetermined reference sample that has been obtained from an individual or individuals without prostate cancer or with benign prostatic hyperplasia).

In another particular example, the subject may be identified as having prostate cancer or as having an increased risk of developing prostate cancer when they have a decreased level of GCNT1 in their blood or urine sample compared to the control sample or the pre-determined reference level (e.g. when the level of GCNT1 protein in their blood or urine sample is lower than the level of GCNT1 protein in the control sample or predetermined reference sample that has been obtained from an individual or individuals without prostate cancer or with benign prostatic hyperplasia).

In another particular example, the subject may be identified as having prostate cancer or as having an increased risk of developing prostate cancer when they have an increased level of FUT8 in their blood or urine sample compared to the control sample or the pre-determined reference level (e.g. when the level of FUT8 protein in their blood or urine sample is higher than the level of FUT8 protein in the control sample or predetermined reference sample that has been obtained from an individual or individuals without prostate cancer or with benign prostatic hyperplasia).

The methods described herein can also be used to distinguish between metastatic and non-metastatic (localised) prostate cancer. In other words, the methods described herein can be used to identify that a subject has metastatic prostate cancer or has an increased risk of developing metastatic prostate cancer. In this context, the control sample used for the comparison step of the method is typically (preferably) obtained from a subject that has non-metastatic, localised, prostate cancer or the pre-determined reference level is the average level of the biomarker in a subject with non-metastatic, localised, prostate cancer. A subject is can then be identified as having metastatic prostate cancer or as having an increased risk of developing metastatic prostate cancer if the comparison step indicates that the subject has one or more of the following: an increased level of ST6GAL1 compared to the (non-metastatic prostate cancer) control sample or the (non-metastatic prostate cancer) pre-determined reference level; an increased level of GCNT1 compared to the (non-metastatic prostate cancer) control sample or the (non-metastatic prostate cancer) pre-determined reference level; an increased level of FUT8 compared to the (non-metastatic prostate cancer) control sample or the (non-metastatic prostate cancer) pre-determined reference level; or an increased level of GALNT7 compared to the (non-metastatic prostate cancer) control sample or the (non-metastatic prostate cancer) pre-determined reference level.

The terms “non-metastatic prostate cancer”, “NON-MET” and “localised prostate cancer” are used interchangeably herein.

In a particular example, the subject may be identified as having metastatic prostate cancer or as having an increased risk of developing metastatic prostate cancer when they have an increased level of FUT8 in their blood or urine sample compared to the control sample or the pre-determined reference level (e.g. when the level of FUT8 protein in their blood or urine sample is higher than the level of FUT8 protein in the control sample or predetermined reference sample that has been obtained from an individual or individuals with localised prostate cancer).

In a particular example, the subject may be identified as having metastatic prostate cancer or as having an increased risk of developing metastatic prostate cancer when they have an increased level of ST6GAL1 in their blood or urine sample compared to the control sample or the pre-determined reference level (e.g. when the level of ST6GAL1 protein in their blood or urine sample is higher than the level of ST6GAL1 protein in the control sample or predetermined reference sample that has been obtained from an individual or individuals with localised prostate cancer).

In a particular example, the subject may be identified as having metastatic prostate cancer or as having an increased risk of developing metastatic prostate cancer when they have an increased level of GCNT1 in their blood or urine sample compared to the control sample or the pre-determined reference level (e.g. when the level of GCNT1 protein in their blood or urine sample is higher than the level of GCNT1 protein in the control sample or predetermined reference sample that has been obtained from an individual or individuals with localised prostate cancer).

In a particular example, the subject may be identified as having metastatic prostate cancer or as having an increased risk of developing metastatic prostate cancer when they have an increased level of GALNT7 in their blood or urine sample compared to the control sample or the pre-determined reference level (e.g. when the level of GALNT7 protein in their blood or urine sample is higher than the level of GALNT7 protein in the control sample or predetermined reference sample that has been obtained from an individual or individuals with localised prostate cancer).

In a specific example, the level of GCNT1, FUT8 or ST6GAL1 is determined in a blood sample of a subject to detect or predict the occurrence of metastatic prostate cancer in the subject (by comparing the levels of GCNT1, FUT8 or ST6GAL1 to the level of the same marker in an equivalent sample from a subject (or e.g. the average of a plurality of subjects) that has non-metastatic, localised, prostate cancer, wherein metastatic prostate cancer is detected or predicted when the level of GCNT1, FUT8 or ST6GAL1 is increased compared to the level of the same markers in the sample from a subject (or e.g. the average of a plurality of subjects) that has non-metastatic, localised, prostate cancer. Optionally, the level of GALNT7 is also determined in the sample.

In another specific example, the level of either GCNT1, FUT8 or ST6GAL1 is determined in a urine sample of a subject to detect or predict the occurrence of metastatic prostate cancer in the subject (by comparing the levels of GCNT1, FUT8 or ST6GAL1 to the level of the same marker in an equivalent sample from a subject (or e.g. the average of a plurality of subjects) that has non-metastatic, localised, prostate cancer, wherein metastatic prostate cancer is detected or predicted when the level of GCNT1, FUT8 or ST6GAL1 is increased compared to the level of the same markers in the sample from a subject (or e.g. the average of a plurality of subjects) that has non-metastatic, localised, prostate cancer. Optionally, the level of GALNT7 is also determined in the sample.

In another specific example, the levels of GCNT1, FUT8 and ST6GAL1 are determined in a blood sample of a subject to detect or predict the occurrence of metastatic prostate cancer in the subject (by comparing the levels of GCNT1, FUT8 and ST6GAL1 to the levels of these markers in an equivalent sample from a subject (or e.g. the average of a plurality of subjects) that has non-metastatic, localised, prostate cancer, wherein metastatic prostate cancer is detected or predicted when the levels of GCNT1, FUT8 and/or ST6GAL1 (preferably GCNT1, FUT8 and ST6GAL1) are increased compared to the levels of these markers in the sample from a subject (or e.g. the average of a plurality of subjects) that has non-metastatic, localised, prostate cancer. Optionally, the level of GALNT7 is also determined in the sample.

In another specific example, the levels of GCNT1, FUT8 and ST6GAL1 are determined in a urine sample of a subject to detect or predict the occurrence of metastatic prostate cancer in the subject (by comparing the levels of GCNT1, FUT8 and ST6GAL1 to the levels of these markers in an equivalent sample from a subject (or e.g. the average of a plurality of subjects) that has non-metastatic, localised, prostate cancer, wherein metastatic prostate cancer is detected or predicted when the levels of GCNT1, FUT8 and/or ST6GAL1 (preferably GCNT1, FUT8 and ST6GAL1) are increased compared to the levels of these markers in the sample from a subject (or e.g. the average of a plurality of subjects) that has non-metastatic, localised, prostate cancer. Optionally, the level of GALNT7 is also determined in the sample.

The term “change” refers in this context to a statistically significant difference in the biomarker level for the sample obtained from the test subject compared to the biomarker levels obtained from the control sample or predetermined reference level. The difference (or change) may be an increase or decrease in biomarker levels compared to the control sample or predetermined reference level.

The terms “decrease”, “decreased” “reduced”, “reduction” or “down-regulated”, “lower” are all used herein generally to mean a decrease by a statistically significant amount. However, for avoidance of doubt, “reduced”, “reduction”, “decreased” or “decrease” means a decrease by at least 10% as compared to a reference level/control, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference/control sample), or any decrease between 10-100% as compared to a reference level/control, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a reference level/control.

The terms “increased”, “increase” or “up-regulated”, “higher” are all used herein to generally mean an increase by a statically significant amount; for the avoidance of any doubt, the terms “increased” or “increase” means an increase of at least 10% as compared to a reference level/control, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level/control, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level/control.

The methods can further comprise selecting, and optionally administering, a treatment regimen for the subject based on the diagnosis (i.e., based on the comparison of the levels of the biomarkers with the reference levels/controls). Treatment can include, for example, surgery (e.g., radical prostectomy) and, in some cases, therapy (e.g., radiation, hormone, ultrasound, chemotherapy, immunotherapy), or combinations thereof. However, in some cases, immediate treatment may not be required, and the subject may be selected for active surveillance.

As used herein, the terms “active surveillance”, “monitoring” and “watchful waiting” are used interchangeably herein to mean closely monitoring a patient's condition without giving any treatment until symptoms appear or change. For example, in prostate cancer, watchful waiting is usually used in older men with other medical problems and early-stage disease.

As used herein, the terms “treat”, “treating” and “treatment” are taken to include an intervention performed with the intention of preventing the development or altering the pathology of a condition, disorder or symptom (i.e. in this case prostate cancer). Accordingly, “treatment” refers to both therapeutic treatment and prophylactic or preventative measures, wherein the object is to prevent or slow down (lessen) the targeted condition, disorder or symptom. “Treatment” therefore encompasses a reduction, slowing or inhibition of the symptoms of prostate cancer, for example of at least 5%, 10%, 20%, 30%, 40%, 50%, 60%, 70%, 80%, 90% or 100% when compared to the symptoms before treatment. In the context of prostate cancer, appropriate treatment may include surgery and/or therapy.

As used herein, the term “surgery” applies to surgical methods undertaken for removal of cancerous tissue, including pelvic lymphadenectomy, radical prostatectomy, transurethral resection of the prostate (TURP), excision, dissection, and tumor biopsy/removal.

As used herein, the term “therapy” includes radiation, hormonal therapy, cryosurgery, chemotherapy, immunotherapy, biologic therapy, and high-intensity focused ultrasound.

The type of treatment will vary depending on the particular form of prostate cancer that the subject has, is suspected of having, is at risk of developing, or is suspected of being at risk of developing.

For example, if the subject has, is suspected of having, is at risk of having, or is suspected of being at risk of having, metastatic prostate cancer, the subject may benefit from treatment with for example androgen deprivation therapy, radiotherapy, immunotherapy. Accordingly, the method may include the step of administering one or more of these treatments to the subject. Other suitable treatments are well known to a person of skill in the art and depend on the specific symptoms of the subject.

Androgens are also closely linked to prostate cancer treatment, with androgen deprivation therapy (ADT) being the principal pharmacological strategy for locally advanced and metastatic disease. ADT utilises drugs to inhibit gonadal and extra-gonadal androgen biosynthesis and competitive AR antagonists to block androgen binding and abrogate AR function. Accordingly, if the subject has, is suspected of having, is at risk of having, or is suspected of being at risk of having, metastatic prostate cancer, a preferred method may include the step of administering androgen deprivation therapy to the subject.

As a further example, if the subject has, is suspected of having, is at risk of having, or is suspected of being at risk of having, non-metastatic, localised, prostate cancer, the subject may benefit from active surveillance or surgery. Accordingly, the method may include the step of administering one or more of these treatments to the subject. Other suitable treatments are well known to a person of skill in the art and depend on the specific symptoms of the subject.

When a therapeutic agent or other treatment is administered, it is administered in an amount and/or for a duration that is effective to treat the prostate cancer or to reduce the likelihood (or risk) of prostate cancer developing in the future. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain analytes (e.g., biomarkers as described herein) that are indicative of prostate cancer. It should be understood that the therapeutic agents described herein are used to treat and/or prevent prostate cancer. Thus, in some cases, they may be used prophylactically in subjects at risk of developing prostate cancer. Thus, in some cases, an effective amount is that amount which can lower the risk of, slow or perhaps prevent altogether the development of prostate cancer. It will be recognized when the therapeutic agent is used in acute circumstances, it is used to prevent one or more medically undesirable results that typically flow from such adverse events. Methods for selecting a suitable treatment, an appropriate dose thereof and modes of administration will be apparent to one of ordinary skill in the art.

The medications or treatments described herein can be administered to the subject by any conventional route, including injection or by gradual infusion over time. The administration may, for example, be by infusion or by intramuscular, intravascular, intracavity, intracerebral, intralesional, rectal, subcutaneous, intradermal, epidural, intrathecal, percutaneous administration. The medications may also be given in e.g. tablet form or in solution. Several appropriate medications and means for administration of the same are well known for treatment of prostate cancer.

The methods described herein are advantageous as they stratify patients into (i) patients that do not have prostate cancer (but may have BPH, prostatitis or an enlarged prostate) and (ii) patients that have prostate cancer. They can also be used to stratify patients into (a) patients that have low grade prostate cancer and (b) patients that have high grade prostate cancer. This allows treatment decisions to be made based on disease stage.

For example, a subject may be identified as having prostate cancer when they have an increased level of ST6GAL1 compared to a control sample (or a pre-determined reference level) from a subject that does not have prostate cancer; an increased level of GALNT7 compared to a control sample (or a pre-determined reference level) from a subject that does not have prostate cancer; an increased level of FUT8 compared to a control sample (or a pre-determined reference level) from a subject that does not have prostate cancer; and/or a decreased level of GCNT1 compared to a control sample (or a pre-determined reference level) from a subject that does not have prostate cancer. Conversely, a subject may be identified as not having prostate cancer when their level(s) of ST6GAL1, GALNT7 and/or FUT8 are not increased compared to the control sample (or the pre-determined reference level); and/or their level of GCNT1 is not decreased compared to the control sample (or the pre-determined reference level).

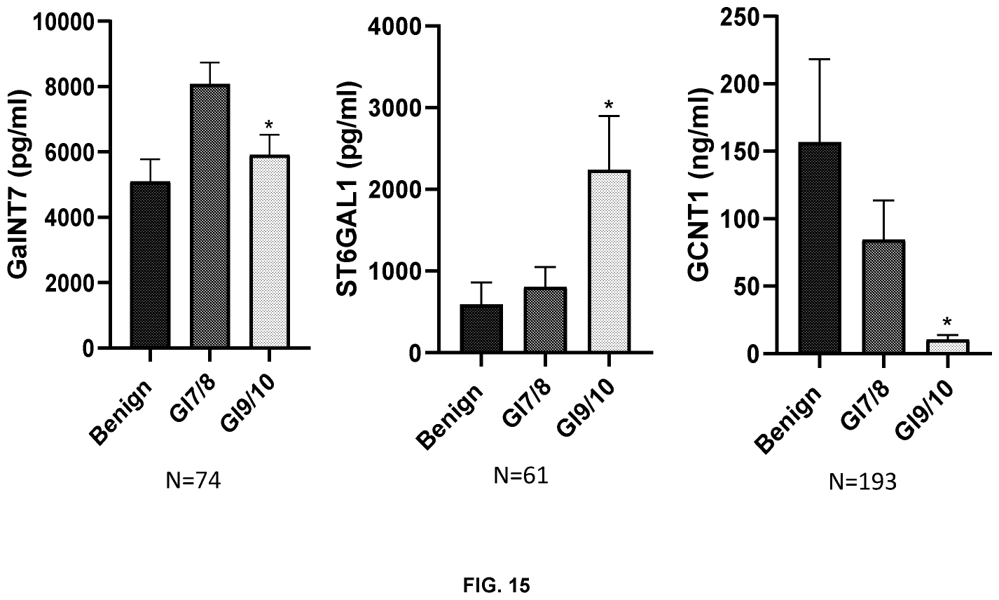

The increase in ST6GAL1, GALNT7 and/or FUT8 levels, and/or decrease in GCNT1 levels may also be used to identify whether the subject has low grade or high grade prostate cancer. Typically, the increase in ST6GAL1, GALNT7 and/or FUT8 levels, and/or decrease in GCNT1 levels is more pronounced (more significant) in subjects with high grade prostate cancer than those with low grade prostate cancer (see for example FIG. 15 herein). ST6GAL1, GALNT7, FUT8 and/or GCNT1 levels can therefore be used to decide the best treatment options for the subject.

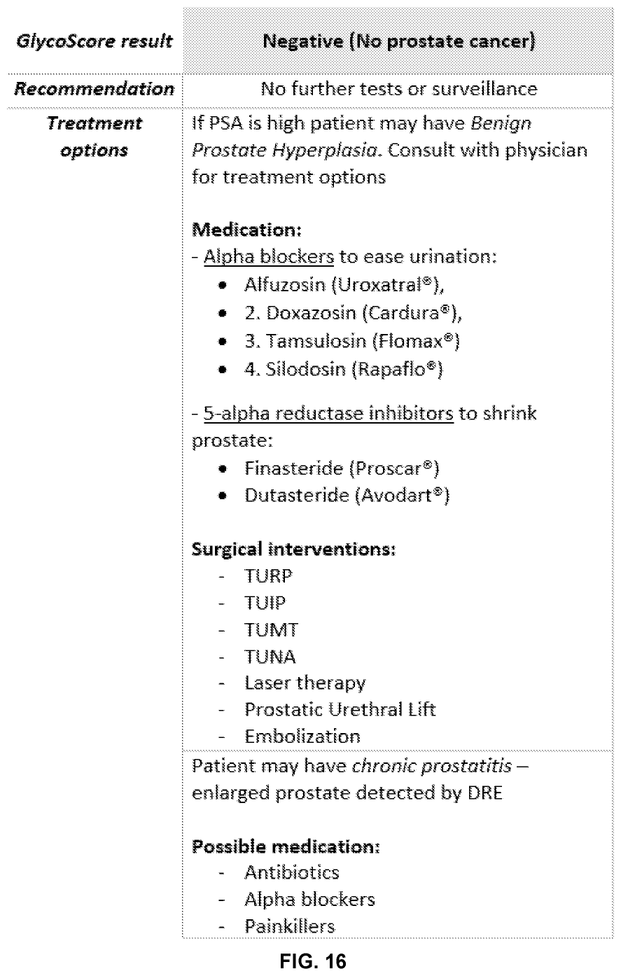

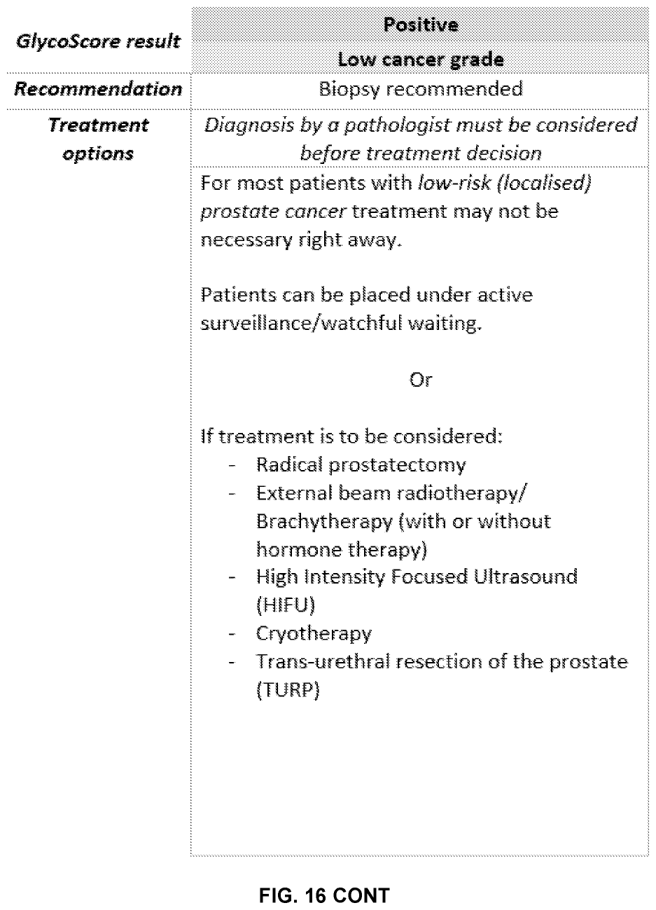

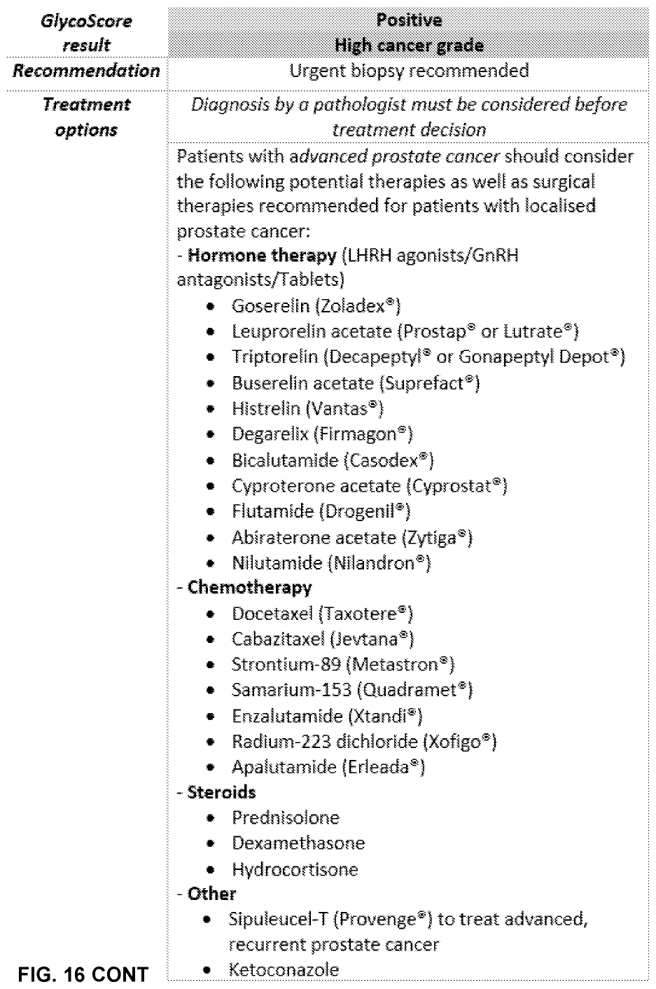

Treatment options for subjects with low grade or high grade prostate cancers are summarised in FIG. 16.

Accordingly, when a subject is identified herein as having (i.e. diagnosed with) prostate cancer, they may be treated with a treatment selected from the group consisting of:

(i) radical prostatectomy;
(ii) external beam radiotherapy/Brachytherapy (with or without hormone therapy);
(iii) High Intensity Focused Ultrasound (HIFU);

### (iv) Cryotherapy;

(v) Trans-urethral resection of the prostate (TURP);
(vi) hormone therapy (e.g. LHRH agonists/GnRH antagonists/Tablets such as Goserelin (Zoladex®), Leuprorelin acetate (Prostap® or Lutrate®), Triptorelin (Decapeptyl® or Gonapeptyl Depot®), Buserelin acetate (Suprefact®), Histrelin (Vantas®), Degarelix (Firmagon®), Bicalutamide (Casodex®), Cyproterone acetate (Cyprostat®), Flutamide (Drogenil®), Abiraterone acetate (Zytiga®), or Nilutamide (Nilandron®)) (vii) Chemotherapy (e.g. Docetaxel (Taxotere®), Cabazitaxel (Jevtana®), Strontium-89 (Metastron®), Samarium-153 (Quadramet®), Enzalutamide (Xtandi®), Radium-223 dichloride (Xofigo®), or Apalutamide (Erleada®))
(viii) Steroids (e.g. Prednisolone, Dexamethasone, Hydrocortisone); and
(ix) Sipuleucel-T (Provenge®) (to treat advanced, recurrent prostate cancer).

For example, when a subject is identified herein as having (i.e. diagnosed with) low grade prostate cancer, they may be placed under active surveillance or be treated with a treatment selected from the group consisting of: radical prostatectomy, external beam radiotherapy/Brachytherapy (with or without hormone therapy), High Intensity Focused Ultrasound (HIFU), Cryotherapy and Trans-urethral resection of the prostate (TURP).

Furthermore, when a subject is identified herein as having (i.e. diagnosed with) high grade and/or metastatic prostate cancer and/or castrate resistant prostate cancer, they may be treated with a treatment selected from the group consisting of:

(i) hormone therapy (e.g. LHRH agonists/GnRH antagonists/Tablets such as Goserelin (Zoladex®), Leuprorelin acetate (Prostap® or Lutrate®), Triptorelin (Decapeptyl® or Gonapeptyl Depot®), Buserelin acetate (Suprefact®), Histrelin (Vantas®), Degarelix (Firmagon®), Bicalutamide (Casodex®), Cyproterone acetate (Cyprostat®), Flutamide (Drogenil®), Abiraterone acetate (Zytiga®), or Nilutamide (Nilandron®)) (ii) Chemotherapy (e.g. Docetaxel (Taxotere®), Cabazitaxel (Jevtana®), Strontium-89 (Metastron®), Samarium-153 (Quadramet®), Enzalutamide (Xtandi®), Radium-223 dichloride (Xofigo®), or Apalutamide (Erleada®))
(iii) Steroids (e.g. Prednisolone, Dexamethasone, Hydrocortisone); and
(iv) Sipuleucel-T (Provenge®) (to treat advanced, recurrent prostate cancer), or Ketoconazole,
optionally in combination with a treatment selected from the group consisting of: radical prostatectomy, external beam radiotherapy/Brachytherapy (with or without hormone therapy), High Intensity Focused Ultrasound (HIFU), Cryotherapy and Trans-urethral resection of the prostate (TURP).
(v) Monoclonal antibody therapies (e.g. Pembrolizumab (keytruda), Avastin (bevacizumab), Erbitux (cetuximab), Rituxan (rituximab) and Herceptin (trastuzumab)).

### Uses

Also provided herein is the use of one or more biomarkers selected from the group consisting of: GALNT7, ST6GAL1, FUT8 and GCNT1 as a biological fluid biomarker for prostate cancer.

In an example, two or three or four of the specified biomarkers may be used.

For example, GALNT7 and ST6GAL1; GALNT7 and GCNT1; or GALNT7 and FUT8 may be used.

GALNT7, ST6GAL1 and GCNT1 may also be used. Alternatively, GALNT7, ST6GAL1 and FUT8 may be used. Alternatively, GALNT7, GCNT1 and FUT8 may be used.

In a further example, ST6GAL1 and GCNT1; or ST6GAL1 and FUT8 may be used. ST6GAL1, GCNT1 and FUT8 may also be used.

In a further example, GCNT1 and FUT8 may be used.

Alternatively, GALNT7, ST6GAL1, GCNT1 and FUT8 may be used.

In a preferred example, GALNT7, ST6GAL1 and GCNT1 (Glycoscore) may be used as biomarkers for prostate cancer generally. In this context “prostate cancer generally” refers to all forms of prostate cancer, including but not limited to localised prostate cancer and metastatic prostate cancer. In addition, FUT8 may also be used.

In an alternative preferred example, FUT8, ST6GAL1 and GCNT1 (metastatic Glycoscore) may be used as biomarkers for metastatic prostate cancer specifically. In addition, GALNT7 may additionally be used.

Any of the above combinations may also be combined with PSA.

Details of the biomarkers, samples, methods, subjects, types of prostate cancer etc are provided elsewhere and apply equally to this aspect.

### Methods for Monitoring Prostate Cancer Progression

An in vitro method for monitoring prostate cancer progression in a subject is also provided herein, the method comprising the steps of:

i) determining the level of one or more biomarker in a biological fluid sample from the subject in accordance with method steps a) to b) of the methods for diagnosing prostate cancer or determining the risk of developing prostate cancer described above; and
ii) repeating step i) for the same subject after a time interval; and
iii) comparing the biomarker levels identified in i) with the biomarker levels identified in ii), wherein a change in the biomarker levels from i) to ii) is indicative of a change in prostate cancer progression in the subject.

The method may be used to monitor the progression of a non-metastatic (localised) or metastatic forms of prostate cancer, amongst others.

Typically, such monitoring methods are performed on subjects that have not yet been treated for prostate cancer (i.e. they have not previously received prostate cancer treatment (therapy or surgery)). Such subjects are described as “naïve” subjects herein.

However, such monitoring methods also encompass methods performed on subjects that have already been treated for prostate cancer. For example, the subject may have previously been diagnosed with metastatic prostate cancer and may have previously received androgen deprivation therapy (ADT). In this example, the methods described herein may be used to monitoring metastatic prostate cancer progression (i.e. relapse, such as relapse into castrate resistant prostate cancer (CRPC)). The biomarkers that are described herein as particularly useful for distinguishing between localised prostate cancer and metastatic prostate cancer (FUT8, GCNT1 and/or ST6GAL1) are particularly useful in this context. Accordingly, the methods described herein may be particularly useful when monitoring for relapse into castrate resistant prostate cancer (especially when the markers FUT8, GCNT1 and/or ST6GAL1 are used as they can distinguish between localised prostate cancer and metastatic prostate cancer (as each of them is increased in metastatic prostate cancer compared to localised prostate cancer).

Monitoring the progression of prostate cancer (and specifically metastatic or non-metastatic forms of prostate cancer) in a subject over time assists in the earliest possible identification of disease progression (e.g. a worsening in disease status or disease symptoms). Such monitoring naturally involves the taking of repeated samples over time. The method may therefore be repeated at one or more time intervals for a particular subject and the results compared to monitor the development, progression or improvement in the prostate cancer (and specifically of the metastatic or non-metastatic forms of prostate cancer) of that subject over time, wherein a change in the amount of level of the one or more biomarker tested for in the biological fluid sample (e.g. blood or urine) is indicative of a change in the progression of the prostate cancer (and specifically the metastatic or non-metastatic forms of prostate cancer) in the subject.

Disease progression (e.g. prostate cancer progression, including the progression of the metastatic or non-metastatic forms of prostate cancer) may be indicated by an increase in the level of GALNT7 detected over time when the results of two or more time intervals are compared for the same subject. In other words, if the method is performed a plurality of times, disease progression may be indicated when the level of GALNT7 detected at the later time interval(s) is higher than that detected at the earlier time interval(s). An “increase” in the level of GALNT7 encompasses detection of GALNT7 at a later time interval when no GALNT7 was detected (i.e. it was not present at detectable levels) when the method was performed previously (i.e. at an earlier time interval) on the same subject (and an equivalent biological fluid sample type). This is particularly relevant when monitoring the progression of prostate cancer in naïve subjects.

Disease progression (e.g. prostate cancer progression, particularly the progression of the metastatic or non-metastatic forms of prostate cancer) may be indicated by an increase in the level of ST6GAL1 detected over time when the results of two or more time intervals are compared for the same subject. In other words, if the method is performed a plurality of times, disease progression may be indicated when the level of ST6GAL1 detected at the later time interval(s) is higher than that detected at the earlier time interval(s). An “increase” in the level of ST6GAL1 encompasses detection of ST6GAL1 at a later time interval when no ST6GAL1 was detected (i.e. it was not present at detectable levels) when the method was performed previously (i.e. at an earlier time interval) on the same subject (and an equivalent biological fluid sample type). This is particularly relevant when monitoring the progression of prostate cancer in naïve subjects.

Disease progression (e.g. prostate cancer progression, particularly the progression of the metastatic or non-metastatic forms of prostate cancer) may be indicated by an increase in the level of FUT8 detected over time when the results of two or more time intervals are compared for the same subject. In other words, if the method is performed a plurality of times, disease progression may be indicated when the level of FUT8 detected at the later time interval(s) is higher than that detected at the earlier time interval(s). An “increase” in the level of FUT8 encompasses detection of FUT8 at a later time interval when no FUT8 was detected (i.e. it was not present at detectable levels) when the method was performed previously (i.e. at an earlier time interval) on the same subject (and an equivalent biological fluid sample type). This is particularly relevant when monitoring the progression of prostate cancer in naïve subjects.

Disease progression (e.g. prostate cancer progression, from no prostate cancer to prostate cancer) may be indicated by a decrease in the level of GCNT1 detected over time when the results of two or more time intervals are compared for the same subject. In other words, if the method is performed a plurality of times, disease progression may be indicated when the level of GCNT1 detected at the later time interval(s) is lower than that detected at the earlier time interval(s). An “decrease” in the level of GCNT1 encompasses no detection of GCNT1 at a later time interval (i.e. it was not present at detectable levels) when GCNT1 was detected when the method was performed previously (i.e. at an earlier time interval) on the same subject (and an equivalent biological fluid sample type). This is particularly relevant when monitoring the progression of prostate cancer in naïve subjects.

Conversely, when monitoring disease progression from non-metastatic to metastatic prostate cancer, disease progression may be indicated by an increase in the level of GCNT1 detected over time when the results of two or more time intervals are compared for the same subject.

In other words, if the method is performed a plurality of times, metastatic prostate cancer disease progression may be indicated when the level of GCNT1 detected at the later time interval(s) is higher than that detected at the earlier time interval(s) (when the patient had non-metastatic prostate cancer. An “increase” in the level of GCNT1 encompasses detection of GCNT1 at a later time interval when GCNT1 was not detected when the method was performed previously (i.e. at an earlier time interval) on the same subject (and an equivalent biological fluid sample type).

Suitable time intervals for monitoring disease progression can easily be identified by a person of skill in the art and will depend on the specific form of prostate cancer (e.g. metastatic or non-metastatic forms of prostate cancer) being monitored. As a non-limiting example, the method may be repeated at least every six months, or at least every year, or whenever clinically needed, i.e. in case of a significant change in prostate cancer symptoms.

Details of the biomarkers, combinations, samples, methods steps, subjects, types of prostate cancer etc are provided elsewhere and apply equally to this aspect.

### Methods for Determining the Therapeutic Effect of a Treatment Regimen for Prostate Cancer

An in vitro method for determining the therapeutic effect of a treatment regimen for prostate cancer is also provided herein, the method comprising:

a) determining the level of one or more biomarker in a biological fluid sample from the subject, wherein the one or more biomarker is selected from the group consisting of GALNT7, ST6GAL1, FUT8 and GCNT1;
b) repeating step a) using a biological fluid sample obtained from the subject after treatment for a time interval; and
c) comparing the level of biomarker determined in step a) to that determined in step b), and identifying that the treatment regimen has a therapeutic effect if one or more of the following is observed: there is a decrease in the level of GALNT7 after treatment; there is a decrease in the level of ST6GAL1 after treatment; there is a decrease in the level of FUT8 after treatment; or there is a change in the level of GCNT1 after treatment.

In one example, the change in level of GCNT1 that is indicative of a therapeutic effect is an increase in GCNT1 level after treatment.

Step a) may first be performed in accordance with the method using a biological fluid sample that was obtained from the subject at a time point before the treatment regimen for prostate cancer began. Alternatively, step a) may first be performed using a biological fluid sample that was obtained from the subject at the same time as commencing the treatment regimen, or at a time point after the treatment regimen for prostate cancer began. The method can therefore be used to determine the therapeutic effect of a treatment regimen for prostate cancer from the outset (i.e. from the start of the regimen) or from a time point after the treatment regimen has started (i.e. determining the therapeutic effect of a treatment regimen for prostate cancer during the treatment regimen itself).

The method can also be useful as a screening tool for determining if specific regimens or treatment modalities have a therapeutic effect on prostate cancer. The tested regimens or treatment modalities may be new regimens or treatment modalities, modified regimens or treatment modalities, or known regimens or treatment modalities that need further testing. In this context, a treatment modality is e.g. a drug or medicament that is useful or suspected to be useful in the treatment of prostate cancer.

Details of the biomarkers, combinations, samples, methods steps, subjects, types of prostate cancer, treatments, etc are provided elsewhere and apply equally to this aspect.

A treatment regimen may be identified as having a therapeutic effect if it results in a delay in disease progression or a delay in the development of symptoms (e.g. over a treatment period). A treatment regimen may also be identified as having a therapeutic effect if it results in an improvement in disease status or symptoms (e.g. over a treatment period). Methods for determining if the treatment regimen has a therapeutic effect are well known in the art.

A treatment period refers to a time interval over which treatment occurs (e.g. 1 month, 3 months, 6 months, 1 year, 2 years, etc).

As an example, an improvement in disease status or symptoms (e.g. over a treatment period) (e.g. improvement in prostate cancer status or symptoms) may be indicated by a decrease in the level of GALNT7 detected over time when the results of two or more time intervals are compared for the same subject. In other words, if the method is performed a plurality of times, an improvement in disease status may be indicated when the level of GALNT7 detected at the later time interval(s) is lower than that detected at the earlier time interval(s). An “decrease” in the level of GALNT7 encompasses no detection of GALNT7 (i.e. it is not present at detectable levels) at a later time interval when GALNT7 was detected when the method was performed previously (i.e. at an earlier time interval) on the same subject (and an equivalent biological fluid sample type).

An improvement in disease status or symptoms (e.g. over a treatment period) may also be indicated by stabilised levels of GALNT7 over time (compared to the level of GALNT7 observed in the absence of treatment over the equivalent time period, or compared to equivalent controls).

As an example, an improvement in disease status or symptoms (e.g. over a treatment period) (e.g. improvement in prostate cancer status or symptoms, particularly the disease status or symptoms of metastatic or non-metastatic forms of prostate cancer) may be indicated by a decrease in the level of ST6GAL1 detected over time when the results of two or more time intervals are compared for the same subject. In other words, if the method is performed a plurality of times, an improvement in disease status may be indicated when the level of ST6GAL1 detected at the later time interval(s) is lower than that detected at the earlier time interval(s). An “decrease” in the level of ST6GAL1 encompasses no detection of ST6GAL1 (i.e. it is not present at detectable levels) at a later time interval when ST6GAL1 was detected when the method was performed previously (i.e. at an earlier time interval) on the same subject (and an equivalent biological fluid sample type).

An improvement in disease status or symptoms (e.g. over a treatment period) may also be indicated by stabilised levels of ST6GAL1 over time (compared to the level of ST6GAL1 observed in the absence of treatment over the equivalent time period, or compared to equivalent controls).

As an example, an improvement in disease status or symptoms (e.g. over a treatment period) (e.g. improvement in prostate cancer status or symptoms, particularly the disease status or symptoms of metastatic or non-metastatic forms of prostate cancer) may be indicated by a decrease in the level of FUT8 detected over time when the results of two or more time intervals are compared for the same subject. In other words, if the method is performed a plurality of times, an improvement in disease status may be indicated when the level of FUT8 detected at the later time interval(s) is lower than that detected at the earlier time interval(s). An “decrease” in the level of FUT8 encompasses no detection of FUT8 (i.e. it is not present at detectable levels) at a later time interval when FUT8 was detected when the method was performed previously (i.e. at an earlier time interval) on the same subject (and an equivalent biological fluid sample type).

An improvement in disease status or symptoms (e.g. over a treatment period) may also be indicated by stabilised levels of FUT8 over time (compared to the level of FUT8 observed in the absence of treatment over the equivalent time period, or compared to equivalent controls).

As would be clear to a person of skill in the art, the direction of change in GCNT1 levels that is indicative of a therapeutic effect may depend on the disease status of the subject prior to treatment and the control/reference used. As a non-limiting example, if the subject has non-metastatic prostate cancer prior to treatment, an increase in GCNT1 levels (e.g. returning to levels equivalent to those observed in a subject with no prostate cancer or with BPH) may be indicative of a therapeutic effect. Other appropriate examples would be clear to a person of skill in the art, in the context of the invention disclosed herein.

An improvement in disease status or symptoms (e.g. over a treatment period) may also be indicated by stabilised levels of GCNT1 over time (compared to the level of GCNT1 observed in the absence of treatment over the equivalent time period, or compared to equivalent controls).

Suitable time intervals for monitoring an improvement in disease status or symptoms (e.g. during treatment of the subject) can easily be identified by a person of skill in the art and will depend on the specific form of prostate cancer (e.g. metastatic or non-metastatic forms of prostate cancer) being monitored. As a non-limiting example, the method may be repeated at least every six months, or at least every year, or at least every two years, or more frequently as required.

Methods for Determining a Subject's Compliance or Adherence with a Prescribed Treatment Regimen for Prostate Cancer

An in vitro method for determining a subject's compliance or adherence with a prescribed treatment regimen for prostate cancer is also provided herein, the method comprising:

a) determining the level of one or more biomarker in a biological fluid sample from the subject, wherein the one or more biomarker is selected from the group consisting of GALNT7, ST6GAL1, FUT8 and GCNT1;
b) repeating step a) after a time interval using a biological fluid sample from the subject after the prescribed start of treatment regimen; and
c) comparing the level of biomarker determined in step a) to that determined in step b), and identifying that the subject has complied or adhered with the prescribed treatment regimen if one or more of the following is observed: there is a decrease in the level of GALNT7 after treatment; there is a decrease in the level of ST6GAL1 after treatment; there is a decrease in the level of FUT8 after treatment; or there is a change in the level of GCNT1 after treatment.

In one example, the change in level of GCNT1 that is indicative of compliance or adherence with the prescribed treatment is an increase in GCNT1 level after treatment.

As would be clear to a person of skill in the art, the direction of change in GCNT1 levels that is indicative of compliance or adherence with the prescribed treatment may depend on the disease status of the subject prior to treatment and the control/reference used. As a non-limiting example, if the subject has non-metastatic prostate cancer prior to treatment, an increase in GCNT1 levels (e.g. returning to levels equivalent to those observed in a subject with no prostate cancer or with BPH) may be indicative of compliance or adherence with the prescribed treatment. Other appropriate examples would be clear to a person of skill in the art, in the context of the invention disclosed herein.

Appropriate subjects, treatments, terminology and permutations or combinations of features have been described in detail above.

The trends for identifying that the subject has complied or adhered with the prescribed treatment regimen are equivalent to those described in detail above in respect of determining the therapeutic effect of a treatment regimen for prostate cancer. This is because a “prescribed treatment regimen” is a recommended treatment regimen and therefore typically has a therapeutic effect (and thus, observation of the therapeutic effect on the biomarker levels is an indication of subject compliance or adherence with the prescribed treatment regimen) Accordingly, all aspects described in detail above for methods for determining the therapeutic effect of a treatment regimen for prostate cancer apply equally here.

### Method of Determining the Clinical Significance of Prostate Cancer

A method of determining the clinical significance of prostate cancer in a subject is also provided herein, the method comprising:

determining the level of one or more biomarker in a biological fluid sample from the subject, wherein the one or more biomarker is selected from the group consisting of: GALNT7, ST6GAL1, FUT8 and GCNT1; and determining therefrom the clinical significance of the prostate cancer.

The methods described herein may be used to differentiate between subjects likely to exhibit normal prostate tissue or Gleason score <6 cytology (e.g. Gleason Grade 1 tissues, which may have a Gleason score of 2 to 4; and some Gleason Grade 2 tissues, which may have a Gleason score of 5 or 6), and those likely to have Gleason score >6 cytology (e.g. Gleason Grade 3-4 tissues, which may have a Gleason score of 7-10). Suitably, the method may be for differentiating between subjects likely to exhibit Gleason score cytology of less than or equal to 8 (which includes low grade prostate cancer cytologies), and those likely to have Gleason score cytology of more than or equal to 9 (high grade prostate cancer cytologies).

A Gleason (GL) score of 6 is generally accepted as the threshold score between benign tissues (having a score of less than or equal to 6) and cancerous tissues (having a score of 6+ or 7+). Low grade prostate cancer typically has GL 6+7 (or GL6 and GL7), whereas high grade prostate cancer typically has GL8+ or GL7(4+3) or more. The term “low grade prostate cancer” therefore encompasses GL 6+7 (or GL6 and GL7), whereas the term “high grade prostate cancer” encompasses GL8+ or GL7(4+3) or more (including GL 9/10).

Details of the relevance of the Gleason score on prostate cancer diagnosis and/or prognosis are provided elsewhere herein and apply equally here.

In one example, the method can be used for diagnosing metastatic prostate cancer or determining the risk of developing metastatic prostate cancer. For example, the method can be used to identify subjects with increased levels of GCNT1 and/or ST6GAL1 in a biological fluid sample (e.g. blood or urine) compared to a subject that has non-metastatic, localised, prostate cancer (or the average level for a plurality of subjects that have non-metastatic, localised, prostate cancer.

In a specific example, the level of either GCNT1 or ST6GAL1 is determined in a blood sample of a subject to detect or predict the occurrence of metastatic prostate cancer in the subject (by comparing the levels of GCNT1 or ST6GAL1 to the level of the same marker in an equivalent sample from a subject (or e.g. the average of a plurality of subjects) that has non-metastatic, localised, prostate cancer, wherein metastatic prostate cancer is detected or predicted when the level of GCNT1 or ST6GAL1 is increased compared to the level of the same markers in the sample from a subject (or e.g. the average of a plurality of subjects) that has non-metastatic, localised, prostate cancer.

In another specific example, the level of either GCNT1 or ST6GAL1 is determined in a urine sample of a subject to detect or predict the occurrence of metastatic prostate cancer in the subject (by comparing the levels of GCNT1 or ST6GAL1 to the level of the same marker in an equivalent sample from a subject (or e.g. the average of a plurality of subjects) that has non-metastatic, localised, prostate cancer, wherein metastatic prostate cancer is detected or predicted when the level of GCNT1 or ST6GAL1 is increased compared to the level of the same markers in the sample from a subject (or e.g. the average of a plurality of subjects) that has non-metastatic, localised, prostate cancer.

In another specific example, the levels of both GCNT1 and ST6GAL1 are determined in a blood sample of a subject to detect or predict the occurrence of metastatic prostate cancer in the subject (by comparing the levels of GCNT1 and ST6GAL1 to the levels of these markers in an equivalent sample from a subject (or e.g. the average of a plurality of subjects) that has non-metastatic, localised, prostate cancer, wherein metastatic prostate cancer is detected or predicted when the levels of GCNT1 and/or ST6GAL1 (preferably both GCNT1 and ST6GAL1) are increased compared to the levels of these markers in the sample from a subject (or e.g. the average of a plurality of subjects) that has non-metastatic, localised, prostate cancer.

In another specific example, the levels of both GCNT1 and ST6GAL1 are determined in a urine sample of a subject to detect or predict the occurrence of metastatic prostate cancer in the subject (by comparing the levels of GCNT1 and ST6GAL1 to the levels of these markers in an equivalent sample from a subject (or e.g. the average of a plurality of subjects) that has non-metastatic, localised, prostate cancer, wherein metastatic prostate cancer is detected or predicted when the levels of GCNT1 and/or ST6GAL1 (preferably both GCNT1 and ST6GAL1) are increased compared to the levels of these markers in the sample from a subject (or e.g. the average of a plurality of subjects) that has non-metastatic, localised, prostate cancer.

The methods described above may include the step of selecting subjects to undergo further investigation and/or selecting subjects for prostate cancer treatment. Examples of appropriate further investigation may include PSA testing and/or prostate tissue histology to identify a Gleason score. Examples of appropriate prostate cancer treatment are discussed elsewhere herein.

Combination with PSA Testing

The PSA blood test is routinely used in the clinic for monitoring and assisting with diagnosis of prostate cancer. Advantageously, the markers identified herein may also be measured in blood. Accordingly, it may be advantageous to combine determining the level of at least one of the markers identified herein (i.e. GALNT7, GCNT1, ST6GAL1 and/or FUT8) with PSA using a biological fluid sample (preferably a blood sample) obtained from the subject in the context of any of the methods described herein.

In one example, a method described herein may determine the level of PSA together with the level of GALNT7 and ST6GAL1. Alternatively, a method described herein may determine the level of PSA together with the level of GALNT7 and GCNT1; or the level of PSA together with the level of GALNT7 and FUT8. Alternatively, a method described herein may determine the level of PSA together with the level of ST6GAL1 and GCNT1; or the level of PSA together with the level of ST6GAL1 and FUT8. In a further example, a method described herein may determine the level of PSA together with the level of GCNT1 and FUT8.

The method may determine the level of PSA together with the level of three additional markers, such as GALNT7, ST6GAL1 and GCNT1. Alternatively, the method may determine the level of PSA together with the level of GALNT7, ST6GAL1 and FUT8. Alternatively, the method may determine the level of PSA together with the level of GALNT7, GCNT1 and FUT8. Alternatively, the method may determine the level of PSA together with the level of ST6GAL1, GCNT1 and FUT8.

Alternatively, the method may determine the level of PSA together with the level of all four additional markers, namely GALNT7, ST6GAL1, GCNT1 and FUT8.

### Kits and Assay Devices

In another aspect, kits are provided for diagnosing prostate cancer or determining the risk of developing prostate cancer in a subject. The kits include reagents suitable for determining levels of a plurality of analytes in a test sample (e.g., reagents suitable for determining levels of the biomarkers disclosed herein).

The kits described herein typically comprise:

(i) a detectably labelled agent that specifically binds to ST6GAL1 protein; and
(ii) one or more of:
a) a detectably labelled agent that specifically binds to GCNT1 protein;
b) a detectably labelled agent that specifically binds to GALNT7 protein; and
c) a detectably labelled agent that specifically binds to FUT8 protein.

Alternatively, the kit may comprise:

(i) a detectably labelled agent that specifically binds to GCNT1 protein; and
(ii) one or more of:
a) a detectably labelled agent that specifically binds to ST6GAL1 protein;
b) a detectably labelled agent that specifically binds to GALNT7 protein; and
c) a detectably labelled agent that specifically binds to FUT8 protein.

Alternatively, the kit may comprise:

(i) a detectably labelled agent that specifically binds to GALNT7 protein; and
(ii) one or more of:
a) a detectably labelled agent that specifically binds to ST6GAL1 protein;
b) a detectably labelled agent that specifically binds to GCNT1 protein; and
c) a detectably labelled agent that specifically binds to FUT8 protein.

Alternatively, the kit may comprise:

(i) a detectably labelled agent that specifically binds to FUT8 protein; and
(ii) one or more of:
d) a detectably labelled agent that specifically binds to ST6GAL1 protein;
e) a detectably labelled agent that specifically binds to GCNT1 protein; and
f) a detectably labelled agent that specifically binds to GALNT7 protein.

Any of the above kits may further comprise a detectably labelled agent that specifically binds to PSA protein.

For example, the kit may comprise a plurality of distinct detectably labelled agents that specifically (independently) bind to GALNT7 and ST6GAL1; GALNT7 and GCNT1; or GALNT7 and FUT8. The kit may comprise a plurality of distinct detectably labelled agents that specifically (independently) bind to GALNT7, ST6GAL1 and GCNT1. Alternatively, kit may comprise a plurality of distinct detectably labelled agents that specifically (independently) bind to GALNT7, ST6GAL1 and FUT8. Alternatively, the kit may comprise a plurality of distinct detectably labelled agents that specifically (independently) bind to GALNT7, GCNT1 and FUT8.

In a further example, the kit may comprise a plurality of distinct detectably labelled agents that specifically (independently) bind to ST6GAL1 and GCNT1; or ST6GAL1 and FUT8. The kit may comprise a plurality of distinct detectably labelled agents that specifically (independently) bind to ST6GAL1, GCNT1 and FUT8.

In a further example, the kit may comprise a plurality of distinct detectably labelled agents that specifically (independently) bind to GCNT1 and FUT8.

Alternatively, the kit may comprise a plurality of distinct detectably labelled agents that specifically (independently) bind to GALNT7, ST6GAL1, GCNT1 and FUT8.

In a preferred example, the kit may comprise a plurality of distinct detectably labelled agents that specifically (independently) bind to GALNT7, ST6GAL1 and GCNT1 (Glycoscore) when the kit is for diagnosing or determining the risk of developing prostate cancer generally.

In an alternative preferred example, the kit may comprise a plurality of distinct detectably labelled agents that specifically (independently) bind to FUT8, ST6GAL1 and GCNT1 (metastatic Glycoscore) when the kit is for diagnosing or determining the risk of developing metastatic prostate cancer specifically.

Any of the above kits may further comprise a detectably labelled agent that specifically binds to PSA protein.

The kits described herein can take on a variety of forms. Typically, the kits will include reagents suitable for determining levels of a plurality of biomarkers (e.g., those disclosed herein, for example GALNT7, ST6GAL1, GCNT1 and FUT8, and optionally PSA) in a sample.

Optionally, the kits may contain one or more control samples or references. Typically, a comparison between the levels of the biomarkers in the subject and levels of the biomarkers in the control samples is indicative of a clinical status (e.g., diagnosis of prostate cancer or risk of developing prostate cancer etc.). Also, the kits, in some cases, will include written information (indicia) providing a reference (e.g., pre-determined values), wherein a comparison between the levels of the biomarkers in the subject and the reference (pre-determined values) is indicative of a clinical status (e.g., diagnosis of prostate cancer or risk of developing prostate cancer etc.). In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

The components of the kit may be housed in a container that is suitable for transportation. Details on the biomarkers is given above and apply equally here. Suitably, the biomarker may be protein.

The term “detectably labelled agent” refers to a binding partner that interacts (i.e. binds) specifically with the biomarker of interest [i.e. GALNT7, ST6GAL1, FUT8, GCNT1, PSA etc] and is also capable of being detected e.g. directly (such as via a fluorescent tag) or indirectly (such as via a labelled secondary antibody). The detectably labelled agent is therefore a selective binding partner for the biomarker of interest (and does not substantially bind to other proteins). Selective binding partners may include antibodies that selectively bind to one of the biomarker of interest.

As used herein, “specifically binds to GALNT7” refers to selective binding of the GALNT7 peptide. Under certain conditions, for example in an immunoassay as described herein, a binding partner that “specifically binds to GALNT7” will selectively bind to this peptide and will not bind in a significant amount to other peptides. Thus the binding partner may bind to GALNT7 with at least 10, 20, 30, 40, 50, 60, 70, 80, 90 or 100 fold more affinity than it binds to a control peptide.

As used herein, “specifically binds to ST6GAL1” refers to selective binding of the ST6GAL1 peptide. Under certain conditions, for example in an immunoassay as described herein, a binding partner that “specifically binds to ST6GAL1” will selectively bind to this peptide and will not bind in a significant amount to other peptides. Thus the binding partner may bind to ST6GAL1 with at least 10, 20, 30, 40, 50, 60, 70, 80, 90 or 100 fold more affinity than it binds to a control peptide.

As used herein, “specifically binds to FUT8” refers to selective binding of the FUT8 peptide. Under certain conditions, for example in an immunoassay as described herein, a binding partner that “specifically binds to FUT8” will selectively bind to this peptide and will not bind in a significant amount to other peptides. Thus the binding partner may bind to FUT8 with at least 10, 20, 30, 40, 50, 60, 70, 80, 90 or 100 fold more affinity than it binds to a control peptide.

As used herein, “specifically binds to GCNT1” refers to selective binding of the GCNT1 peptide. Under certain conditions, for example in an immunoassay as described herein, a binding partner that “specifically binds to GCNT1” will selectively bind to this peptide and will not bind in a significant amount to other peptides. Thus the binding partner may bind to GCNT1 with at least 10, 20, 30, 40, 50, 60, 70, 80, 90 or 100 fold more affinity than it binds to a control peptide.

As used herein, “specifically binds to PSA” refers to selective binding of the PSA peptide. Under certain conditions, for example in an immunoassay as described herein, a binding partner that “specifically binds to PSA” will selectively bind to this peptide and will not bind in a significant amount to other peptides. Thus the binding partner may bind to PSA with at least 10, 20, 30, 40, 50, 60, 70, 80, 90 or 100 fold more affinity than it binds to a control peptide.

In some examples the kits include the detectably labelled agent(s) on a continuous (e.g. solid) surface, such as a lateral flow surface. Alternatively, in examples comprising more than one detectably labelled agent, the detectably labelled agent(s) may be located in distinct (i.e. spatially separate) zones on a (e.g. solid) surface, such as a multiwall micro-titre plate (e.g. for an ELISA assay). Other appropriate surfaces and containers that are well known in the art may also form part of the kits described herein.

In one example, the kit further comprises one or more reagents for detecting the detectably labelled agent. Suitable reagents are well known in the art and include but are not limited to standard reagents and buffers required to perform any one of the appropriate detection methods that may be used (and are well known in the art). In one example, the kit comprises one or more of the following: a multi-well plate, ball bearing(s), extraction buffer, extraction bottle and a lateral flow device lateral flow device.

An assay device is also provided for diagnosing prostate cancer or determining the risk of developing prostate cancer in a subject.

Typically, the device comprises a surface with at least two detectably labelled agents located thereon, wherein the at least two detectably labelled agents are:

(i) a detectably labelled agent that specifically binds to ST6GAL1 protein; and
(ii) one or more of:
a) a detectably labelled agent that specifically binds to GCNT1 protein;
b) a detectably labelled agent that specifically binds to GALNT7 protein; and
c) a detectably labelled agent that specifically binds to FUT8 protein.

Alternatively, the device may comprise a surface with at least two detectably labelled agents located thereon, wherein the at least two detectably labelled agents are:

(i) a detectably labelled agent that specifically binds to GCNT1 protein; and
(ii) one or more of:
d) a detectably labelled agent that specifically binds to ST6GAL1 protein;
e) a detectably labelled agent that specifically binds to GALNT7 protein; and
f) a detectably labelled agent that specifically binds to FUT8 protein.

Alternatively, the device may comprise a surface with at least two detectably labelled agents located thereon, wherein the at least two detectably labelled agents are:

(i) a detectably labelled agent that specifically binds to GALNT7 protein; and
(ii) one or more of:
d) a detectably labelled agent that specifically binds to ST6GAL1 protein;
e) a detectably labelled agent that specifically binds to GCNT1 protein; and
f) a detectably labelled agent that specifically binds to FUT8 protein.

Alternatively, the device may comprise a surface with at least two detectably labelled agents located thereon, wherein the at least two detectably labelled agents are:

(i) a detectably labelled agent that specifically binds to FUT8 protein; and
(ii) one or more of:
g) a detectably labelled agent that specifically binds to ST6GAL1 protein;
h) a detectably labelled agent that specifically binds to GCNT1 protein; and
i) a detectably labelled agent that specifically binds to GALNT7 protein.

Any of the above devices may further comprise a detectably labelled agent that specifically binds to PSA protein.

For example, the device may comprise a plurality of distinct detectably labelled agents that specifically (independently) bind to GALNT7 and ST6GAL1; GALNT7 and GCNT1; or GALNT7 and FUT8. The device may comprise a plurality of distinct detectably labelled agents that specifically (independently) bind to GALNT7, ST6GAL1 and GCNT1. Alternatively, device may comprise a plurality of distinct detectably labelled agents that specifically (independently) bind to GALNT7, ST6GAL1 and FUT8. Alternatively, the device may comprise a plurality of distinct detectably labelled agents that specifically (independently) bind to GALNT7, GCNT1 and FUT8.

In a further example, the device may comprise a plurality of distinct detectably labelled agents that specifically (independently) bind to ST6GAL1 and GCNT1; or ST6GAL1 and FUT8. The device may comprise a plurality of distinct detectably labelled agents that specifically (independently) bind to ST6GAL1, GCNT1 and FUT8.

In a further example, the device may comprise a plurality of distinct detectably labelled agents that specifically (independently) bind to GCNT1 and FUT8.

Alternatively, the device may comprise a plurality of distinct detectably labelled agents that specifically (independently) bind to GALNT7, ST6GAL1, GCNT1 and FUT8.

In a preferred example, the device may comprise a plurality of distinct detectably labelled agents that specifically (independently) bind to GALNT7, ST6GAL1 and GCNT1 (Glycoscore) when the device is for diagnosing or determining the risk of developing prostate cancer generally.

In an alternative preferred example, the device may comprise a plurality of distinct detectably labelled agents that specifically (independently) bind to FUT8, ST6GAL1 and GCNT1 (metastatic Glycoscore) when the device is for diagnosing or determining the risk of developing metastatic prostate cancer specifically.

Any of the above devices may further comprise a detectably labelled agent that specifically binds to PSA protein.

The at least two detectably labeled agents may be located in separate zones on the surface. In other words, the at least two detectably labelled agents may be located in distinct (i.e. spatially separate) zones on a (e.g. solid) surface, such as a multiwell micro-titre plate.

Detectably labelled agent(s) that specifically bind to the biomarker(s) of interest are described in detail elsewhere herein.

The assay device comprises a surface upon which the detectably labelled agents are located. Appropriate surfaces include a continuous (e.g. solid) surface, such as a lateral flow surface, a dot blot surface, a dipstick surface or a surface suitable for performing surface plasmon resonance. Other appropriate surfaces include microtitre plates, multi-well plates etc. Other appropriate surfaces that are well known in the art may also form part of the assay device described herein.

Appropriate assay device formats therefore include but are not limited to device formats suitable for performing any one of lateral flow, dot blot, ELISA, or surface plasmon resonance assays for detecting the presence, level or absence of the biomarker of interest.

### Additional Methods of the Invention

A method is also provided herein, comprising:

(a) preparing a substantially cell-free plasma or serum sample;
(b) diluting the plasma or serum sample of step (a) by at least 5 fold to generate a diluted plasma or serum sample;
(c) measuring the amount of one or more of level of one or more of protein selected from the group consisting of: ST6GAL1, GALNT7, GCNT1 and FUT8 in the diluted plasma or serum sample.

A method for preparing a substantially cell-free plasma or serum sample for analysing the amount of one or more of protein selected from the group consisting of: ST6GAL1, GALNT7, GCNT1 and FUT8 in a subject is also provided herein, comprising:

(a) preparing a substantially cell-free plasma or serum sample;
(b) diluting the plasma or serum sample of step (a) by at least 5 fold to generate a diluted plasma or serum sample;
(c) measuring the amount of one or more of level of one or more of protein selected from the group consisting of: ST6GAL1, GALNT7, GCNT1 and FUT8 in the diluted plasma or serum sample.

Step (b) may comprise diluting the plasma or serum sample of step (a) by about 5 to about 7 fold.

A substantially cell-fee serum sample is typically prepared from a whole blood sample, by allowing the blood to clot at ambient temperature (e.g. between 15° C. and 25° C.) and then removing the clot by centrifugation at about 1000 to 2000×g for about 10 minutes. The centrifugation is preferably performed in a refrigerated centrifuge.

Accordingly, a method is provided herein, comprising:

(a) allowing a whole blood sample to clot at ambient temperature to generate a clotted sample;
(b) centrifuging the clotted sample of (a) at about 1000 to about 2000×g for at least about 10 minutes;
(c) obtaining a substantially cell-free serum sample from the centrifuged sample of step (b); and
(c) measuring the amount of one or more of level of one or more of protein selected from the group consisting of: ST6GAL1, GALNT7, GCNT1 and FUT8 in the substantially cell-free serum sample.

A method for preparing a substantially cell-free serum sample for analysing the amount of one or more of protein selected from the group consisting of: ST6GAL1, GALNT7, GCNT1 and FUT8 in a subject is also provided herein, comprising:

(a) allowing a whole blood sample to clot at ambient temperature to generate a clotted sample;
(b) centrifuging the clotted sample of (a) at about 1000 to 2000×g for at least about 10 minutes;
(c) obtaining a substantially cell-free serum sample from the centrifuged sample of step (b); and
(c) measuring the amount of one or more of level of one or more of protein selected from the group consisting of: ST6GAL1, GALNT7, GCNT1 and FUT8 in the substantially cell-free serum sample.

A substantially cell-fee plasma sample is typically prepared from a whole blood sample by collecting the blood into an anti-coagulant-treated tube and then removing cells by centrifugation at about 1000 to 2000×g for about 10 minutes. The centrifugation is preferably performed in a refrigerated centrifuge.

Accordingly, a method is provided herein, comprising:

(a) contacting a whole blood sample with an anti-coagulant-treated tube;
(b) centrifuging the tube of (a) at about 1000 to about 2000×g for at least about 10 minutes;
(c) obtaining a substantially cell-free plasma sample from the centrifuged sample of step (b); and
(c) measuring the amount of one or more of level of one or more of protein selected from the group consisting of: ST6GAL1, GALNT7, GCNT1 and FUT8 in the substantially cell-free plasma sample.

A method for preparing a substantially cell-free plasma sample for analysing the amount of one or more of protein selected from the group consisting of: ST6GAL1, GALNT7, GCNT1 and FUT8 in a subject is also provided herein, comprising:

(a) contacting a whole blood sample with an anti-coagulant-treated tube;
(b) centrifuging the tube of (a) at about 1000 to about 2000×g for at least about 10 minutes;
(c) obtaining a substantially cell-free plasma sample from the centrifuged sample of step (b); and
(c) measuring the amount of one or more of level of one or more of protein selected from the group consisting of: ST6GAL1, GALNT7, GCNT1 and FUT8 in the substantially cell-free plasma sample.

Urine samples obtained from a subject may also be prepared before the amount of one or more protein selected from the group consisting of: ST6GAL1, GALNT7, GCNT1 is measured/analysed.

Accordingly, a method is provided herein, comprising:

(a) centrifuging a urine sample obtained from a subject at about 1500 relative centrifugal force for at least about 15 minutes;
(c) obtaining a substantially cell-free urine sample from the centrifuged sample of step (b); and
(c) measuring the amount of one or more of level of one or more of protein selected from the group consisting of: ST6GAL1, GALNT7, GCNT1 and FUT8 in the substantially cell-free urine sample.

A method for preparing a substantially cell-free urine sample for analysing the amount of one or more of protein selected from the group consisting of: ST6GAL1, GALNT7, GCNT1 and FUT8 in a subject is also provided herein, comprising:

(a) centrifuging a urine sample obtained from a subject at about 1500 relative centrifugal force for at least about 15 minutes;
(c) obtaining a substantially cell-free urine sample from the centrifuged sample of step (b); and
(c) measuring the amount of one or more of level of one or more of protein selected from the group consisting of: ST6GAL1, GALNT7, GCNT1 and FUT8 in the substantially cell-free urine sample.

Samples that are “substantially cell-free” are samples from which most (and ideally all) cells have been removed. A substantially cell-free plasma or serum sample means that the percentage of cells in the sample is significantly lower than that found in the biological sample (e.g. the unprocessed sample, or the pre-cleared sample e.g. whole blood sample) from which the plasma or serum sample has been generated. Typically, the percentage of cells in a substantially cell-free plasma or serum sample is less than 10%, less than 5%, less than 4%, less than 3%, less than 2%, less than 1% etc of the plasma or serum sample. Ideally there are no detectable cells in the plasma or serum sample. Similarly, a substantially cell-free urine sample means that the percentage of cells in the sample is significantly lower than that found in the biological sample (e.g. the unprocessed sample, or the pre-cleared sample e.g. urine sample obtained directly from the subject) from which the substantially cell-free urine sample has been generated. Typically, the percentage of cells in a substantially cell-free urine sample is less than 10%, less than 5%, less than 4%, less than 3%, less than 2%, less than 1% etc of the urine sample.

Use of a substantially cell-free plasma or serum sample or a substantially cell-free urine sample in the methods described herein is advantageous, as it reduces or eliminates detection of intracellular ST6GAL1, GALNT7, GCNT1 and FUT8.

Data storage aspects Biomarker levels and/or reference levels may be stored in a suitable data storage medium (e.g., a database) and are, thus, also available for future diagnoses. This also allows efficiently diagnosing prevalence for a disease because suitable reference results can be identified in the database once it has been confirmed (in the future) that the subject from which the corresponding reference sample was obtained did have prostate cancer. As used herein a “database” comprises data collected (e.g., analyte and/or reference level information and/or patient information) on a suitable storage medium. Moreover, the database, may further comprise a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative of prostate cancer (e.g. a query search). Thus, if an identical or similar data set can be identified in the data collection, the test data set will be associated with prostate cancer. Consequently, the information obtained from the data collection can be used to diagnose prostate cancer or based on a test data set obtained from a subject. More preferably, the data collection comprises characteristic values of all analytes comprised by any one of the groups recited above.

The methods described herein may further include communication of the results or diagnoses (or both) to technicians, physicians or patients, for example. In certain examples, computers will be used to communicate results or diagnoses (or both) to interested parties, e.g., physicians and their patients.

In some examples, the results or diagnoses (or both) are communicated to the subject as soon as possible after the diagnosis is obtained. The results or diagnoses (or both) may be communicated to the subject by the subject's treating physician. Alternatively, the results or diagnoses (or both) may be sent to a subject by email or communicated to the subject by phone. A computer may be used to communicate the results or diagnoses by email or phone. In certain examples, the message containing results or diagnoses may be generated and delivered automatically to the subject using a combination of computer hardware and software which will be familiar to artisans skilled in telecommunications.

### Companion Diagnostic

The methods kits, assay devices and uses provided herein may be used as part of a companion diagnostic e.g. as part of a medical device, often an in vitro device, which provides information that is essential for the safe and effective use of a corresponding drug or biological product (wherein the corresponding drug or biological product is for treating or preventing prostate cancer).

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wley and Sons, N Y (1994); and Hale and Marham, The Harper Collins Dictionary of Biology, Harper Perennial, N.Y. (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Example 1: Detection of Glycosylation Enzymes in Prostate Cancer Tissue Samples

Using the methods outlined in Munkley et al., 2016, the inventors have previously identified several glycosylation enzymes that are upregulated in prostate cancer tissue at the RNA level.

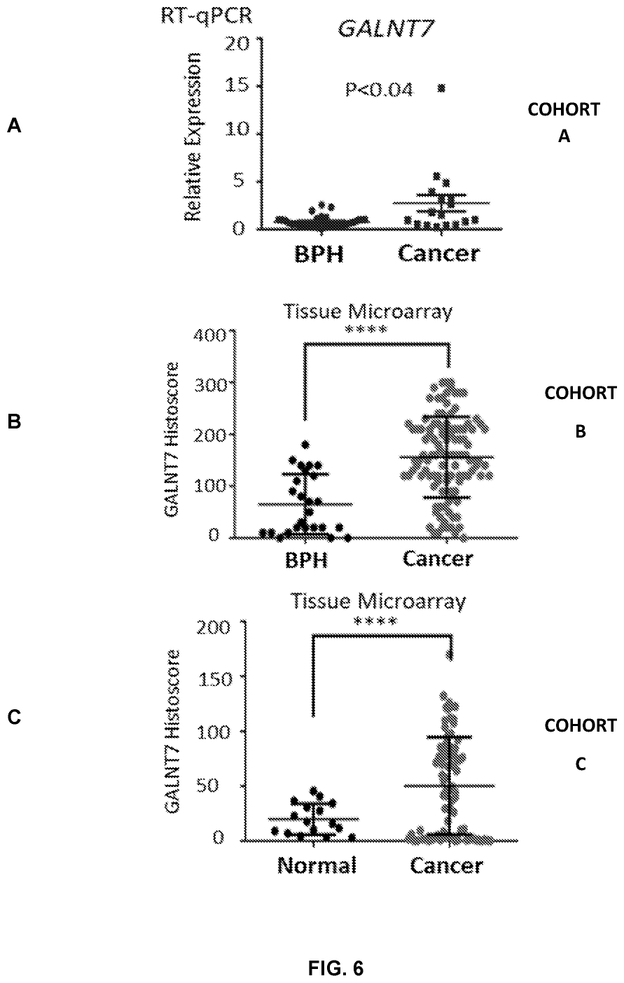

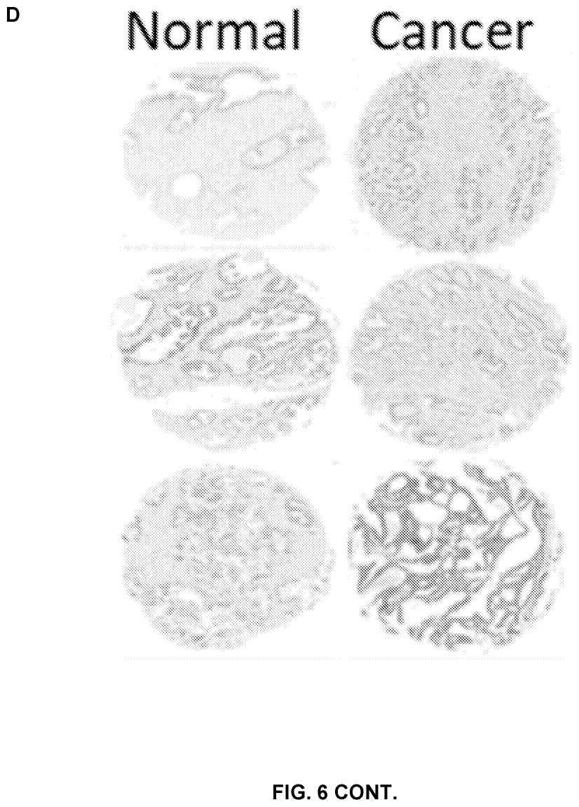

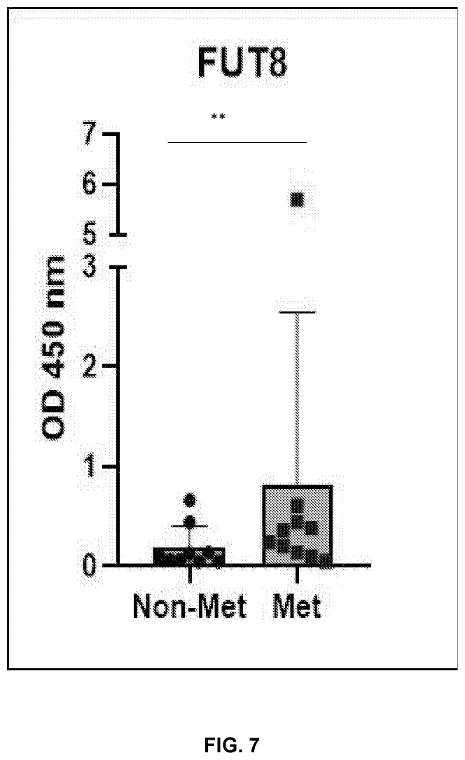

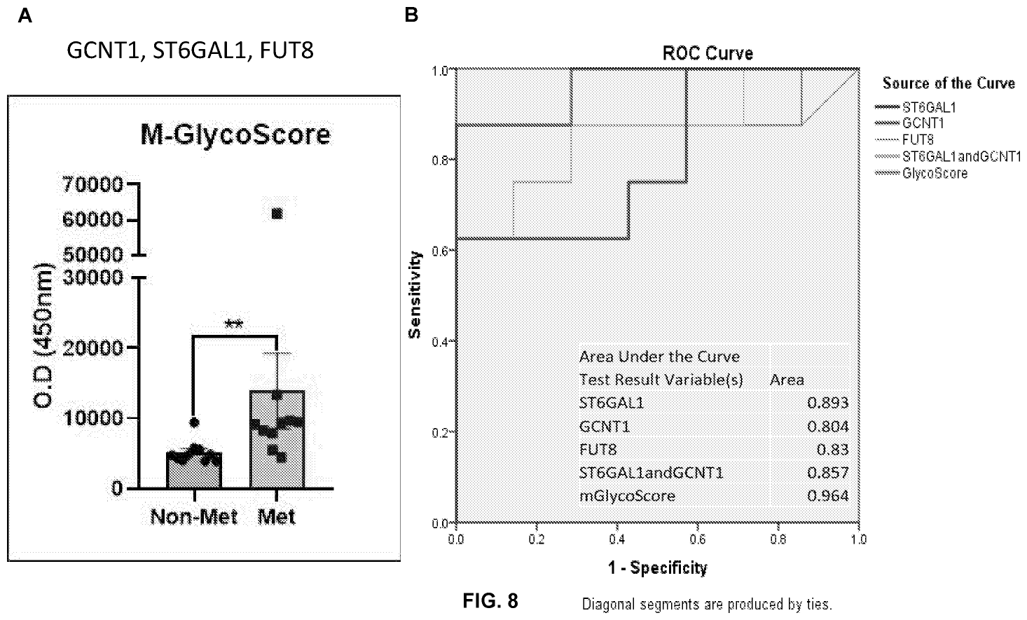

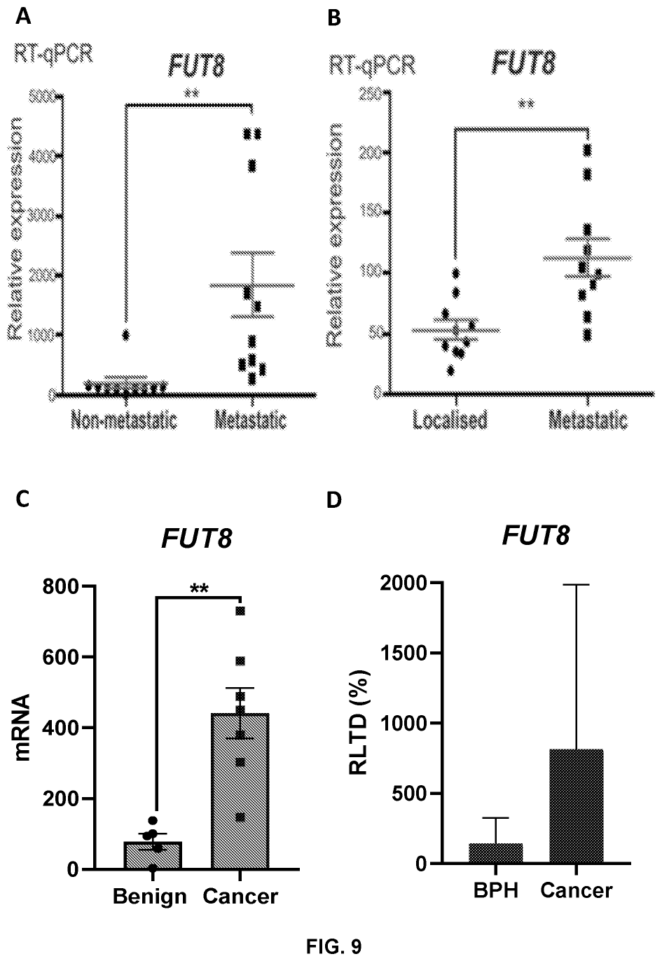

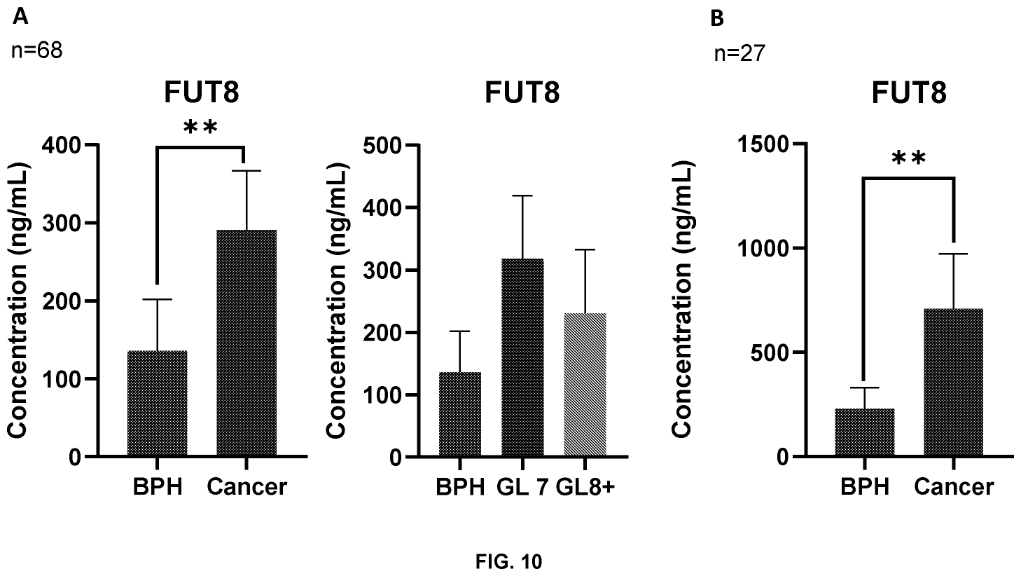

The inventors have now also studied glycosylation enzyme protein levels and have shown that that GALNT7 protein is upregulated in prostate cancer tissue in multiple patient cohorts (FIG. 6).

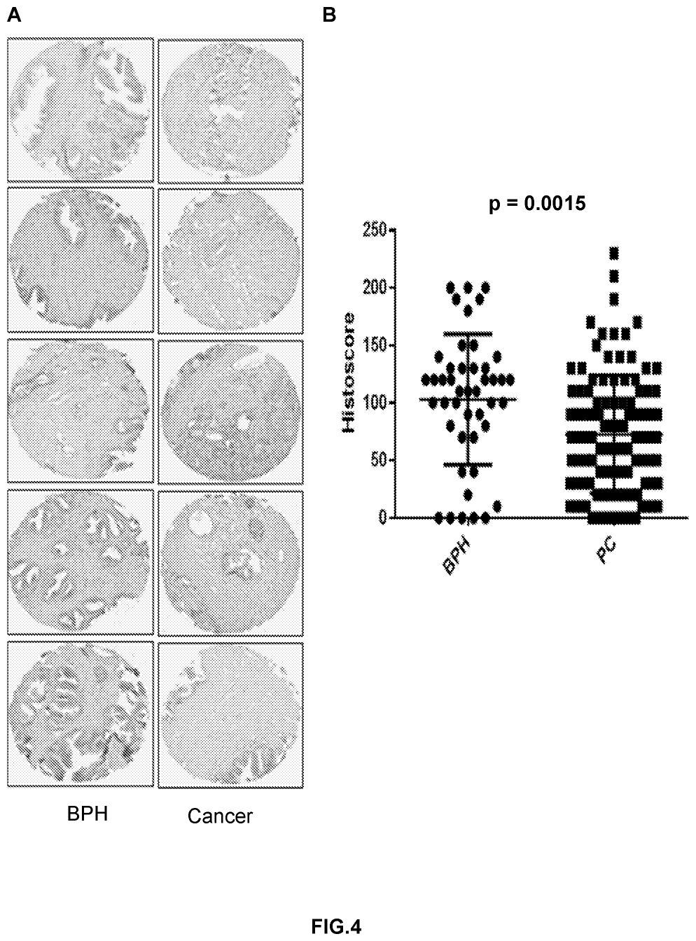

The inventors have also studied ST6GAL1 protein levels in cancer tissue and compared it to benign tissue using immunohistochemistry (IHC) staining. The data shown in FIG. 4 indicates that levels of ST6GAL1 protein are reduced in cancer tissue compared to benign tissue. This is contrary to the data presented in Munkley et al., 2016, which suggested that ST6GAL1 levels were higher in cancerous tissue (based on RNA data). This suggests that the levels of RNA do not correlate with the levels of protein for ST6GAL1. This could be for a number of reasons e.g. how stable the protein is, or because ST6GAL1 is being secreted.

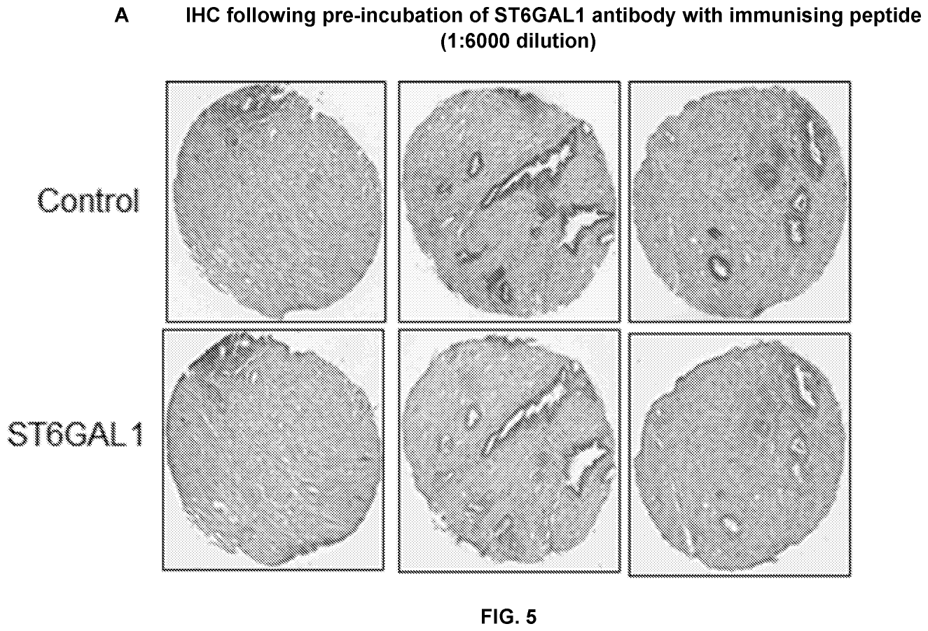

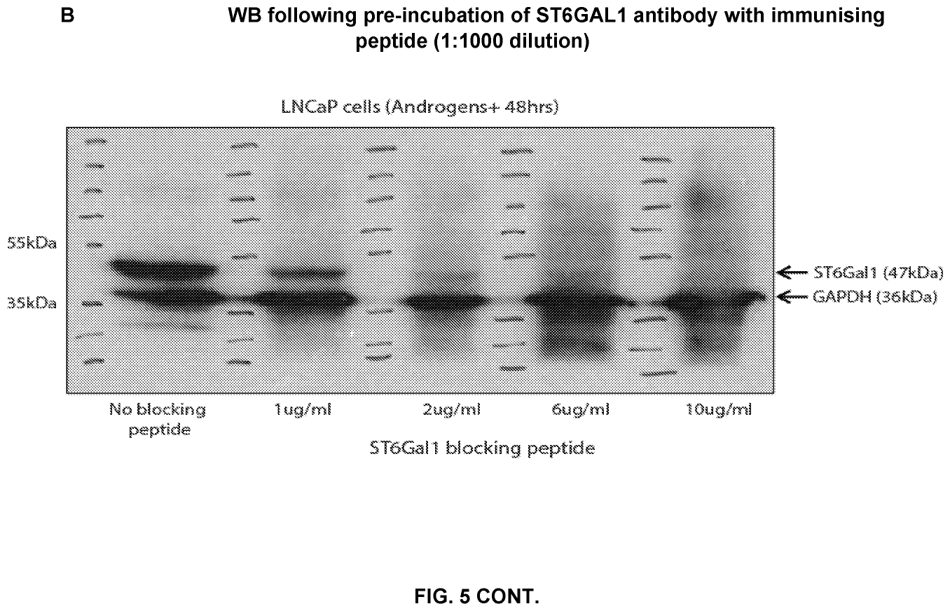

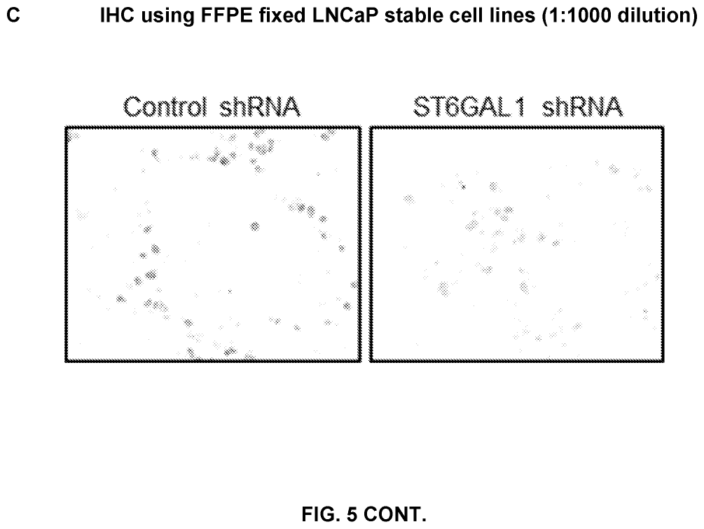

To ensure that the data presented in FIG. 4 herein is accurate, the inventors validated the antibody (previously validated and published by the inventors for use in western blot, Munkley et al., 2016), for IHC as well (FIG. 5). The data shown therein confirms that the chosen ST6GAL1 antibody works well by IHC. It also confirms that, using IHC, ST6GAL1 protein levels are reduced in cancer compared to benign tissue.

Glycosylation enzymes therefore show differential expression patterns in prostate cancer tissue versus benign tissue. Due to their fundamental role in the regulation of glycoprotein and glycolipid biosynthesis, glycosylation enzymes are localised within the Golgi apparatus and endoplasmic reticulum, and remain within the intracellular compartment of the cell.

The inventors have surprisingly found that certain glycosylation enzymes (GCNT1, GALNT7, FUT8 and ST6GAL1) are secreted into biological fluids such as blood and urine in males with prostate cancer (see below). These glycosylation enzymes may therefore advantageously be used as biomarkers for prostate cancer.

### Example 2: Detection of GCNT1, GALNT7, FUT8 and ST6GAL1 in Biological Fluid Samples from Subjects with Prostate Cancer

The inventors have analysed the levels of GCNT1, GALNT7, FUT8 and ST6GAL1 individually, and in combination, in blood and/or urine.

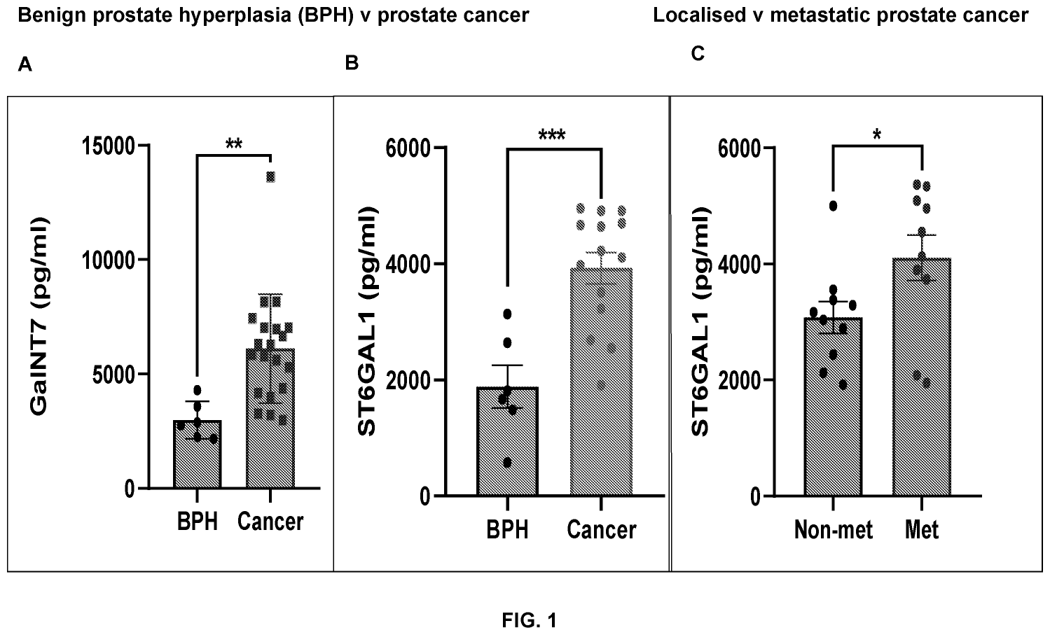

The inventors have shown herein that the glycosylation enzymes GALNT7 and ST6GAL1 are upregulated in the blood of men with prostate cancer (FIG. 1). The enzymes are detected herein using a simple sandwich ELISA test, however, any other appropriate method may also be used.

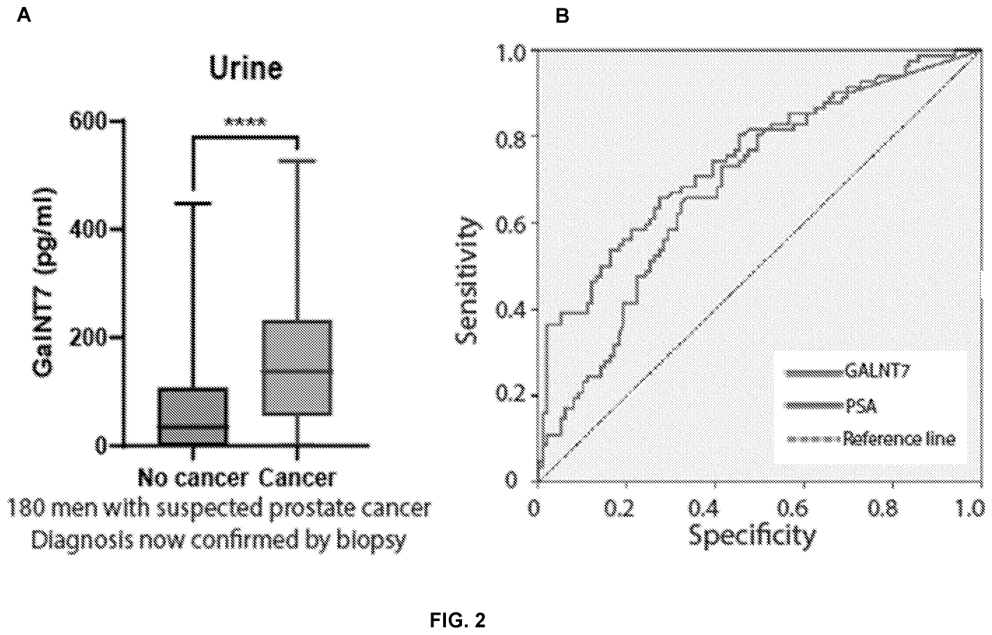

FIG. 2 shows that these glycosylation enzyme biomarkers are more accurate in distinguishing between prostate cancer and non-cancer than PSA, even when one glycosylation enzyme is analysed on its own (in this example, GALNT7, the upper line in FIG. 2B, is measured on its own in urine and compared to PSA; see also Table 1).

TABLE 1
GALNT7 can distinguish BPH and prostate
cancer more accurately than PSA.

| | Area under curve (AUC) |
|---|---|
| GALNT7 | 0.78* |
| PSA | 0.69 |

| | Area under curve |
|---|---|
| GALNT7: | 0.78** |
| PSA: | 0.69 |

The cohort used to generate these data contains 180 urine samples from men with suspected prostate cancer—diagnosis has since been confirmed by biopsy for each patient (prostate cancer diagnosis YES/NO and low/high grade). The samples were blind tested, before calculating the AUC for PSA and GALNT7.

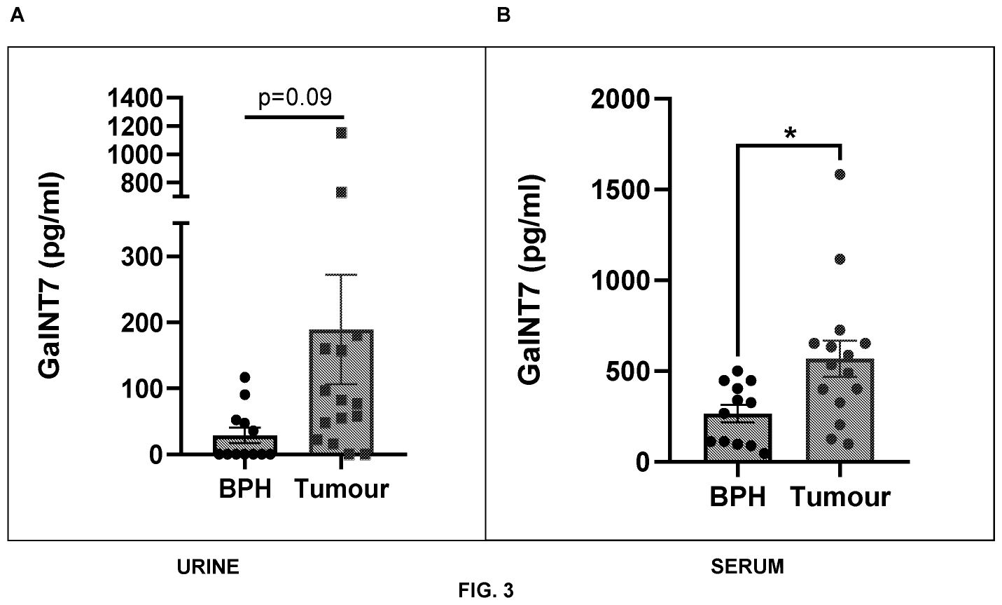

The inventors have shown that prostate cancer not only promotes secretion of GALNT7 and ST6GAL1 into blood, but also promotes secretion of these enzymes into urine. FIG. 3 shows a comparison of GALNT7 levels in plasma and urine samples from the same patients and demonstrates that GALNT7 can be detected in both blood and urine from the same patient, with the highest levels being found in blood.

### Example 3: Validation of a Non-Invasive Diagnostic Test to Distinguish Benign and Aggressive Prostate Cancer Using the Combination of GALNT7 and ST6GAL1 and Comparison with PSA

The inventors have demonstrated that a panel of three glycosylation enzymes are upregulated in the blood and urine of men with prostate cancer and this can be detected using a simple sandwich ELISA test. The glycosylation enzymes GALNT7 and ST6GAL1 are upregulated in the blood of men with prostate cancer whilst GCNT1 protein levels are decreased, and this can be detected using a simple sandwich ELISA test (FIG. 1A, B). The combined levels of these enzymes (GlycoScore) can be used diagnostically to differentiate benign prostatic hyperplasia (BPH) and prostate cancer more accurately than PSA, and can further identify non-metastatic and metastatic patients (for which PSA has no value) (see for example FIG. 1 C).

The extracapsular extension can also be predicted (where the cancer has spread outside of the prostate) with >90% accuracy in urine (data not shown).

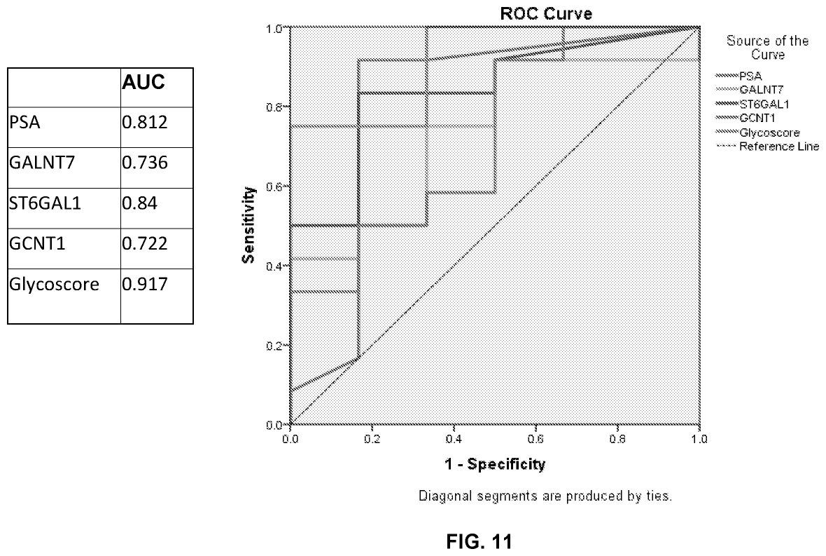

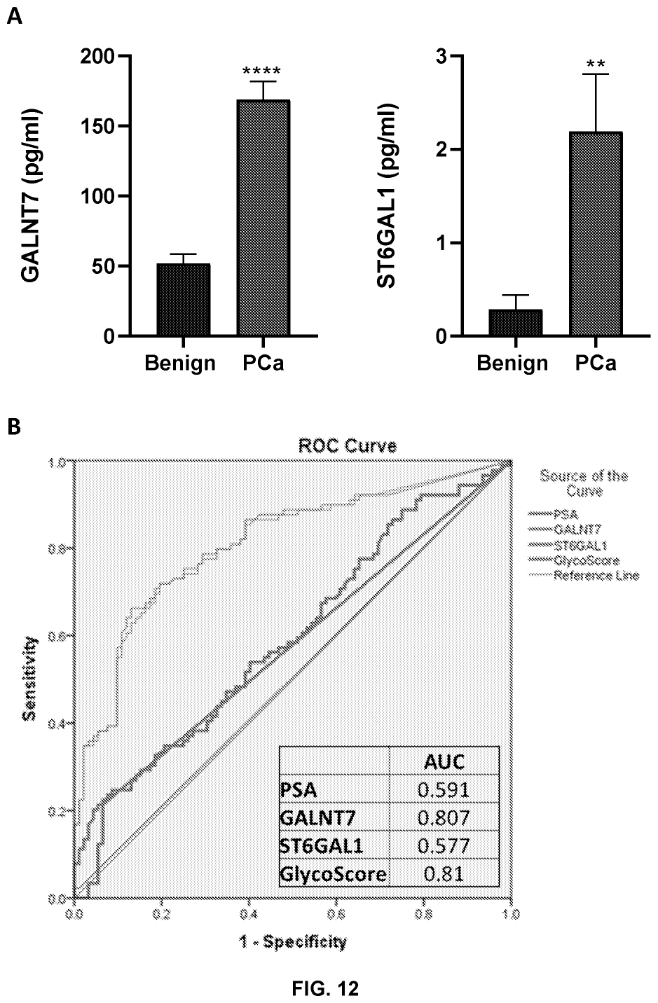

The GlycoScore and metastatic Glycoscore tests have substantial commercial potential, as they address a known un-met clinical need and would also likely reduce costs by limiting the number of biopsies needed and treating patients with the highest risk potential. FIG. 11 shows that diagnostic capacity of the three glycosylation in enzymes, PSA, and the three glycosylation enzymes combined (Glycoscore). These were measured in the serum of a small discovery cohort of patients with histologically confirmed BPH or prostate cancer. Although the AUC for two of the enzymes (GALNT7 and GCNT1) is not greater than that of PSA, the AUC for ST6GAL1 is greater. The AUC for the Glycoscore is the highest, at 0.917, showing that the combined levels of these enzymes gives the greatest diagnostic power to distinguish between BPH and aggressive prostate cancer.

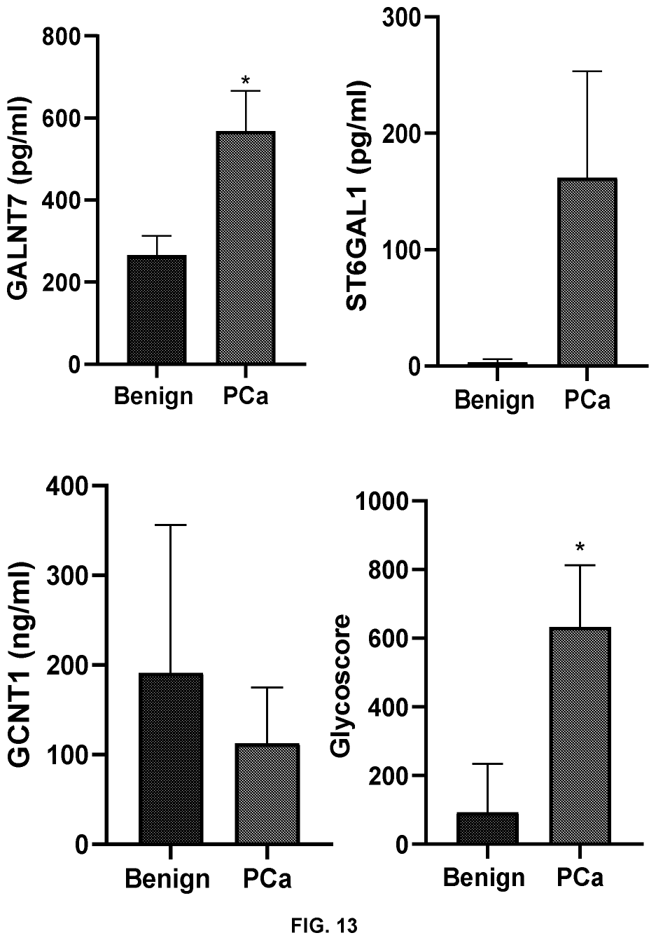

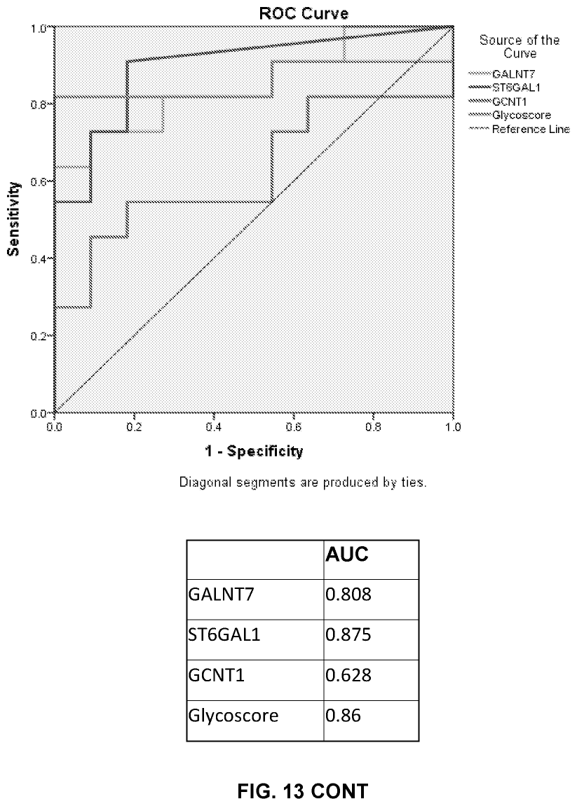

FIG. 13 shows that in the serum of an independent cohort of 27 patients with either ‘no cancer’ or prostate cancer, the levels of the individual levels of GALNT7 and ST6GAL1 have good predictive power. However, the combined levels of these enzymes have an AUC of 0.86, showing that Glycoscore can distinguish between those patients with, and without prostate cancer.

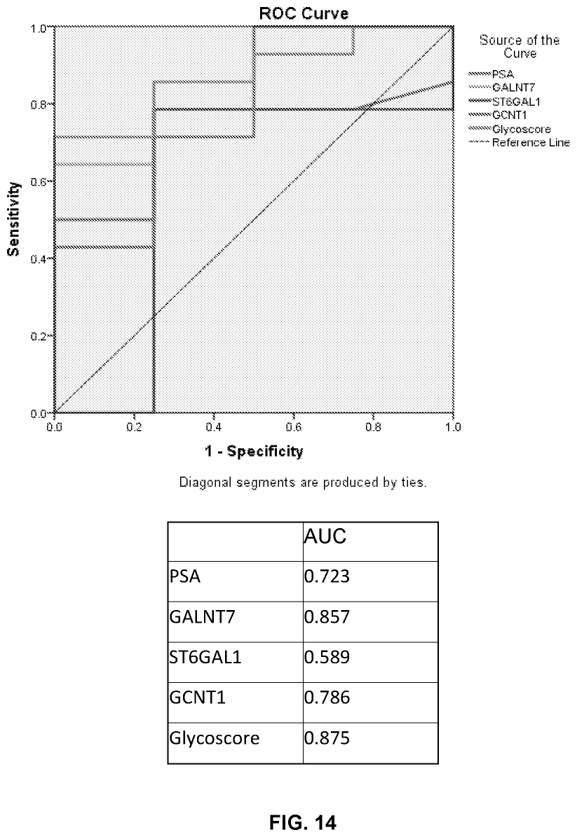

FIG. 14 shows the diagnostic capacity of these three glycosylation enzymes, PSA and the Glycoscore, as serum based biomarkers in a larger validatory cohort of pateints with either BPH or aggressive prostate cancer. The data confirms that the combined levels of these enzymes, the Glycoscore, has the greatest AUC (0.875) compared with an AUC of 0.723 for PSA, confirming that the Glycoscore has a greater capacity to distinguish between BPH and aggressive prostate cancer.

### Materials and Methods

### Enzyme Detection

The inventors initial studies were performed using low cost dot-blotting techniques. To develop this further the inventors sourced and validated custom-made ELISA kits (which have higher specificity and can be easily used in hospital labs). These have been used to produce the data in FIGS. 1 to 3.

Serum samples were generated from whole blood as follows. After collection of the whole blood, allow the blood to clot by leaving it undisturbed at ambient temperature. This usually takes 15-30 minutes. Remove the clot by centrifuging at 1,000-2,000×g for about 10 minutes in a refrigerated centrifuge. The resulting supernatant is designated serum. Following centrifugation, it is important to immediately transfer the liquid component (serum) into a clean polypropylene tube using a Pasteur pipette. The samples should be maintained at ˜2-8° C. while handling. If the serum is not analysed immediately, the serum should be apportioned into aliquots, stored, and transported at −20° C. or lower. It is important to avoid freeze-thaw cycles because this is detrimental to many serum components.

Plasma samples were generated from whole blood as follows. Collect whole blood into anticoagulant-treated tubes. Cells are removed from plasma by centrifugation for 10 minutes at 1,000-2,000×g using a refrigerated centrifuge. Centrifugation for 15 minutes at 2,000×g depletes platelets in the plasma sample. The resulting supernatant is designated plasma. Following centrifugation, it is important to immediately transfer the liquid component (plasma) into a clean polypropylene tube using a Pasteur pipette. The samples should be maintained at 2-8° C. while handling. If the plasma is not analysed immediately, the plasma should be apportioned into aliquots, stored, and transported at −20° C. or lower. It is important to avoid freeze-thaw cycles.

Samples were generated from urine as follows. Freshly collected urine was processed by centrifugation at 1500 relative centrifugal force at 4° C. for 15 minutes with no brake. Supernatant was divided into 15 ml centrifuge tubes and frozen at −80° C.

For the ELISA experiments, serum was thawed on ice and diluted 7-fold (for GALNT7, ST6GAL1 and GCNT1) or 5-fold (for measuring FUT8) in the provided assay diluent. Urine was thawed on ice and centrifuged at 2000 rpm (>500 g) for 10 minutes at 4° C. prior to the assay. All samples were kept on ice throughout the assay. Glycosylation enzyme levels in human urine and serum were detected by sandwich ELISA. The following ELISA kits were purchased to determine enzyme levels, GALNT7 (RayBiotech, Q86SF2), ST6GAL1 (Abcam, ab243669), GCNT1 (RayBiotech, Q02742) and FUT8 (RayBiotech, ELH-FUT8). All samples and standards were assayed in duplicate according to each manufacturer's protocol.

For GALNT7, GCNT1 and FUT8, 100 μl of standard or sample was added to the appropriate wells of the provided 96-well plate (pre-coated with enzyme specific antibody). Samples were incubated for 2.5 hours at room temperate on an orbital shaker. Samples were then removed and the wells washed four times with 300 μl 1× wash buffer (provided). Plates were then inverted and blotted against clean paper. 100 μl of biotinylated antibody (against specific glycosylation enzymes) was then added and incubated for 1 hour at room temperature with gentle shaking. The solution was then discarded and wells washed four times with 300 μl 1× wash buffer. Plates were then inverted and blotted against clean paper. 100 μl of streptavidin (provided) was then added to each well and incubated at room temperature on an orbital shaker for 45 minutes. The solution was then discarded and wells washed four times with 300 μl 1× wash buffer. Plates were then inverted and blotted against clean paper. 100 μl of TMB One-Step substrate reagent (provided) was then added to each well and the plate incubated for 30 minutes at room temperature, in the dark, on an orbital shaker. 50 μl of stop solution (provided) was then added to each well. The assay was then read using a Thermo Fisher Varioskan Lux microplate reader. Absorbance was ready at 450 nm.

To measure ST6GAL1, 50 μl of standard or samples was added to the appropriate wells of the provided 96-well plate (pre-coated with enzyme specific antibody). 50 μl of antibody cocktail (provided) was also added to each well. The plate was then incubated for 1 hour at room temperature on an orbital shaker. The solution was then discarded and wells washed three times with 350 μl 1× wash buffer (provided). Plates were then inverted and blotted against clean paper. 100 μl of TMB development solution (provided) was then added to each well, and the plate incubated for 10 minutes at room temperature in the dark on an orbital shaker. 100 μl of stop solution (provided) was then added to each well and incubated for 1 minute. The assay was read using a Thermo Fisher Varioskan Lux microplate reader. Absorbance was ready at 450 nm.

Protein concentrations for each sample were interpolated from the standard curve using GraohaPad Prism 8 and then adjusted for dilution factors. The enzyme levels were then combined to generate a GlycoScore or metastatic Glyco-Score for each patient. Receiver operator curves (ROC) for the individual enzymes, GlycoScore and PSA values were generated using SPSS to determine the area under the curve and ROC coordinate points. As GCNT1 is a negative predictor of disease, values for GCNT1 were inverted prior to generating ROC curves. Once ROC coordinate points were obtained, the were used to identify a GlycoScore cut off value for the GlycoScore.

To calculate the sensitivity/specificity of the test urine levels of each enzyme can be calculated and multivariate analysis of covariance (MANCOVA) and combined ROC curve analysis can be performed.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

Scott E & Munkley J. Glycans as biomarkers in prostate cancer. IJMS 2019, 20(6), 1389. Munkley, J. Glycosylation is a global target for androgen control in prostate cancer cells. Endocrine Related Cancer (2017) 24 (3), R49-R64.
Munkley J et al. Glycosylation is an Androgen-Regulated Process Essential for Prostate Cancer Cell Viability. eBioMedicine 2016; 8:103-16.
Munkley J & Elliott D J. Hallmarks of glycosylation in cancer. Oncotarget 2016 7(23):35478-89.
Munkley J, Mills I G, Elliott D J. The role of glycans in the development and progression of prostate cancer. Nature Reviews Urology 2016 (6):324-33. doi: 10.1038/nrurol.2016.65.
Munkley J et al. The androgen receptor controls expression of the sTn antigen and cell adhesion through induction of ST6GalNAc1 in prostate cancer. Oncotarget 2015, 6(33), 34358-3437.
Stanley P, Golgi glycosylation; Cold Spring Harb Perspect Biol. 2011 Apr. 1; 3(4)). The American Joint Committee on Cancer (AJCC), AJCC Cancer Staging Manual (7th Ed., 2010)
Heitzer, E., Haque, I. S., Roberts, C. E. S. et al. Current and future perspectives of liquid biopsies in genomics-driven oncology. Nat Rev Genet 20, 71-88 (2019).
Litwin M S and Tan H J., The Diagnosis and Treatment of Prostate Cancer: A Review. JAMA. 2017 Jun. 27; 317(24):2532-254.
Chughtai et al. Benign prostatic hyperplasia. Nat Rev Dis Primers. 2016 May 5; 2:16031.

1. An in vitro method for diagnosing prostate cancer or determining the risk of developing prostate cancer in a subject, the method comprising the steps of:
a) determining the level of one or more biomarker in a biological fluid sample from the subject, wherein the one or more biomarker is selected from the group consisting of: ST6GAL1, GALNT7, FUT8 and GCNT1;
b) comparing the level of the one or more biomarker with the level of the same biomarker in a control sample or with a pre-determined reference level for the same biomarker; and
c) identifying a subject as having prostate cancer or as having an increased risk of developing prostate cancer if the comparison in step b) indicates that the subject has one or more of the following: an increased level of ST6GAL1 compared to the control sample or the pre-determined reference level; an increased level of GALNT7 compared to the control sample or the pre-determined reference level; an increased level of FUT8 compared to the control sample or the pre-determined reference level; a decreased level of GCNT1 compared to the control sample or the pre-determined reference level.

2. The method of claim 1, wherein the control sample is from a control subject that does not have prostate cancer, optionally wherein the control sample is from a subject that has benign prostatic hyperplasia, prostatitis or an enlarged prostate.

3. The method of claim 1, wherein the pre-determined reference level is the average level of the biomarker in a control subject that does not have prostate cancer, optionally wherein the pre-determined reference level is the average level of the biomarker in a subject that has benign prostatic hyperplasia, prostatitis or an enlarged prostate.

4. An in vitro method for diagnosing metastatic prostate cancer or determining the risk of developing metastatic prostate cancer in a subject, the method comprising the steps of:
a) determining the level of one or more biomarker in a biological fluid sample from the subject, wherein the one or more biomarker is selected from the group consisting of: ST6GAL1, FUT8, GCNT1 and GALNT7;
b) comparing the level of the one or more biomarker with the level of the same biomarker in a control sample or with a pre-determined reference level for the same biomarker, wherein the control sample is from a subject that has non-metastatic, localised, prostate cancer or the pre-determined reference level is the average level of the biomarker in a subject with non-metastatic, localised, prostate cancer; and
c) identifying a subject as having metastatic prostate cancer or as having an increased risk of developing metastatic prostate cancer if the comparison in step b) indicates that the subject has one or more of the following: an increased level of ST6GAL1 compared to the control sample or the pre-determined reference level; an increased level of GCNT1 compared to the control sample or the pre-determined reference level; an increased level of FUT8 compared to the control sample or the pre-determined reference value; or an increased level of GALNT7 compared to the control sample or the pre-determined reference level.

5. The method of claim 4, wherein the biological fluid sample is blood or urine.

6. The method of claim 4, wherein step a) comprises determining the level of at least two or three the recited biomarkers in the biological fluid sample.

7. The method of claim 4, wherein step
a) comprises determining the level of:
ST6GAL1 and GALNT7;
ST6GAL1 and GCNT1;
ST6GAL1, GCNT1 and GALNT7;
ST6GAL1, GCNT1 and FUT8; or
ST6GAL1, GCNT1, GALNT7 and FUT8;
in the biological fluid sample.

8. The method of claim 4, wherein the subject is a human.

9. The method of claim 4, wherein the level of biomarker is determined at the protein level, optionally using a process selected from: ELISA assay, immunoblotting, lateral flow assay, protein microarray and mass spectrometry.

10. The method of claim 4, further comprising selecting a treatment regimen for the subject based on the comparison of the level of the biomarker with the control sample or with the pre-determined reference level.

11. The method of claim 10, further comprising administering the selected treatment regimen to the subject, optionally wherein the selected treatment regimen comprises surgery, radiotherapy, chemotherapy, immunotherapy, hormone therapy, ultrasound therapy, or combinations thereof.

12. The method of claim 4, wherein the method further comprises determining the level of PSA in the biological fluid sample.

13. Use of one or more biomarkers selected from the group consisting of: ST6GAL1, GALNT7, FUT8 and GCNT1 as a biological fluid biomarker for prostate cancer.

14. The use according to claim 13, wherein the use is for distinguishing between non-metastatic, localised, prostate cancer and metastatic prostate cancer.

15. The use according to claims 13 to 14, wherein the biomarkers are:
ST6GAL1 and GALNT7;
ST6GAL1 and GCNT1;
ST6GAL1, GCNT1 and GALNT7;
ST6GAL1, GCNT1 and FUT8; or
ST6GAL1, GCNT1, GALNT7 and FUT8.

16. The use according to claims 13 to 15, wherein PSA is used as an additional biomarker.

17. An in vitro method for monitoring prostate cancer progression in a subject, the method comprising the steps of:
i) determining the level of one or more biomarker in a biological fluid sample from the subject in accordance with method steps a) to b) of claim 1; and
ii) repeating step i) for the same subject after a time interval; and
iii) comparing the biomarker levels identified in i) with the biomarker levels identified in ii),
wherein a change in the biomarker levels from i) to ii) is indicative of a change in prostate cancer progression in the subject.

18. The in vitro method of claim 17, wherein the method is for monitoring for relapse into castrate resistant prostate cancer.

19. An in vitro method for determining the therapeutic effect of a treatment regimen for prostate cancer, the method comprising:
a) determining the level of one or more biomarker in a biological fluid sample from the subject, wherein the one or more biomarker is selected from the group consisting of ST6GAL1, GALNT7, FUT8 and GCNT1;
b) repeating step a) using a biological fluid sample obtained from the subject after treatment for a time interval; and
c) comparing the level of biomarker determined in step a) to that determined in step b), and identifying that the treatment regimen has a therapeutic effect if one or more of the following is observed: there is a decrease in the level of ST6GAL1 after treatment; there is a decrease in the level of GALNT7 after treatment; there is a decrease in the level of FUT8 after treatment; or there is a change in the level of GCNT1 after treatment.

20. An in vitro method for determining a subject's compliance or adherence with a prescribed treatment regimen for prostate cancer, the method comprising:
a) determining the level of one or more biomarker in a biological fluid sample from the subject, wherein the one or more biomarker is selected from the group consisting of ST6GAL1, GALNT7, FUT8 and GCNT1;
b) repeating step a) after a time interval using a biological fluid sample from the subject after the prescribed start of treatment regimen; and
c) comparing the level of biomarker determined in step a) to that determined in step b), and identifying that the subject has complied or adhered with the prescribed treatment regimen if one or more of the following is observed: there is a decrease in the level of ST6GAL1 after treatment; there is a decrease in the level of GALNT7 after treatment; there is a decrease in the level of FUT8 after treatment; or there is a change in the level of GCNT1 after treatment.

21. The method of claim 19, wherein the treatment comprises surgery, radiotherapy, chemotherapy, immunotherapy, hormone therapy, ultrasound therapy, or combinations thereof.

22. The method of claim 19, wherein the biological fluid sample is blood or urine.

23. The method of claim 19, wherein the level of at least two biomarkers selected from the group consisting of: ST6GAL1, GALNT7, FUT8 and GCNT1 is determined in the biological fluid sample.

24. The method of claim 23, wherein the level of:
ST6GAL1 and GALNT7;
ST6GAL1 and GCNT1;
ST6GAL1, GCNT1 and GALNT7;
ST6GAL1, GCNT1 and FUT8; or
ST6GAL1, GCNT1, GALNT7 and FUT8
is determined in the biological fluid sample.

25. The method of claim 19, wherein the subject is a human.

26. The method of claim 19, wherein the level of biomarker is determined at the protein level, optionally using a process selected from the group consisting of immunoblotting, lateral flow assay, ELISA assay, protein microarray and mass spectrometry.

27. The method of claim 19, wherein the method further comprises determining the level of PSA in the biological fluid sample.

28. A method of determining the clinical significance of prostate cancer in a subject, the method comprising:
determining the level of one or more biomarker in a biological fluid sample from the subject, wherein the one or more biomarker is selected from the group consisting of: ST6GAL1, GALNT7, FUT8 and GCNT1; and determining therefrom the clinical significance of the prostate cancer.

29. The method of claim 28, wherein the method is for differentiating between subjects likely to exhibit normal prostate tissue or Gleason score <6 cytology, and those likely to have Gleason score >6 cytology.

30. The method of claim 28, wherein the method is for diagnosing metastatic prostate cancer or determining the risk of developing metastatic prostate cancer.

31. The method of claim 28, comprising the step of selecting subjects to undergo further investigation and/or selecting subjects for prostate cancer treatment.

32. (canceled)

33. (canceled)

34. (canceled)

35. (canceled)

36. (canceled)

37. (canceled)
